(19) United States
(12) Patent Application Publication
(10) Pub. No.: US 2005/0267133 A1
(43) Pub. Date: Dec. 1, 2005

(54) Pyrazolopyrimidines as kinase inhibitors

(76) Inventors: Matthew Lee Brown, Indianapolis, IN (US); Mui Cheung, Durham, NC (US); Scott Howard Dickerson, Durham, NC (US); David Harold Drewry, Durham, NC (US); Karen Elizabeth Lackey, Durham, NC (US); Andrew James Peat, Durham, NC (US); Stephen Andrew Thomson, Durham, NC (US); James Marvin Veal, Apex, NC (US); Jayme Lyn Roark Wilson, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/521,910

(22) PCT Filed: Jul. 21, 2003
(86) PCT No.: PCT/US03/22717

§ 371 Date: Jan. 20, 2005

Related U.S. Application Data
(60) Provisional application No. 60/397,898, filed on Jul. 23, 2002.

(51) Int. Cl. A61K 31/519; C07D 487/02

(52) U.S. Cl. 514/262.1; 544/262

(57) ABSTRACT

The present invention relates generally to inhibitors of the kinases and more particularly to novel pyrazolopyrimidine compounds

### FIELD OF THE INVENTION

The present invention relates generally to inhibitors of the kinases, such as GSK3 or TIE2, and more particularly to pyrazolopyrimidine compounds useful as kinase inhibitors.

### BACKGROUND OF THE INVENTION

The present invention provides compounds that are useful pharmacological agents for any disease states mediated, for example alleviated through the inhibition or antagonism, of protein kinases. In particular, the present invention relates to compounds that demonstrate protein tyrosine kinase and/or protein serine/threonine kinase inhibition.

The protein kinases represent a large family of proteins which play a central role in the regulation of a wide variety of cellular processes and maintaining control over cellular function (Hanks, et al., Science, 1988, 241, 42-52). The loss of control over cellular regulation can often lead to aberrant cell function or death, often resulting in a disease state in the parent organism. A partial list of such kinases includes ab1, ATK, bcr-ab1, Blk, Brk, Btk, c-kit, c-met, c-src, CDK1, CDK2, CDK4, CDK6, cRaf1, CSF1R, CSK, EGFR, ErbB2, ErbB3, ErbB4, ERK, Fak, fes, FGFR1, FGFR2, FGFR3, FGFR4, FGFR5, Fgr, FLK-4, fit-1, Fps, Frk, Fyn, GSK3, Hck, IGF-1R, INS-R, Jak, KDR, Lck, Lyn, MEK, p38, PDGFR, PIK, PKC, PYK2, ros, TIE1, TIE2, TRK, Yes, and Zap70. Examples of kinase therapy include, but should not be limited to: (1) inhibition of c-Src (Brickell, Critical Reviews in Oncogenesis 1992, 3, 401-46; Courtneidge, Seminars in Cancer Biology 1994, 5, 239-46), raf (Powis, Pharmacology [symbol] Therapeutics 1994, 62, 57-95) and the cyclin-dependent kinases (CDKs) 1, 2 and 4 in cancer (Pines, Current Opinion in Cell Biology 1992, 4,144-8; Lees, Current Opinion in Cell Biology 1995, 7,773-80; Hunter and Pines, Cell 1994, 79, 573-82), (2) inhibition of CDK2 or PDGF-R kinase in restenosis (Buchdunger, et al., Proceedings of the National Academy of Science USA 1995, 92, 2258-62), (3) inhibition of CDK5 and GSK3 kinases for Alzheimer's (Hosoi, et al., Journal of Biochemistry (Tokyo) 1995, 117, 741-9; Aplin, et al., Journal of Neurochemistry 1996, 67, 699-707), (4) inhibition of c-Src kinase in osteoporosis (Tanaka, et al., Nature 1996, 383, 528-31), (5) inhibition of GSK-3 kinase in type-2 diabetes (Borthwick, et al., Biochemical Et Biophysical Research Communications 1995, 210, 738-45), discussed in more detail below; (6) inhibition of the p38 kinase for inflammation (Badger, et al., The Journal of Pharmacology and Experimental Therapeutics 1996, 279, 1453-61); (7) inhibition of VEGF-R1-3 and TIE-1 and -2 kinases in diseases which involve angiogenesis (Shawver, et al., Drug Discovery Today 1997, 2, 50-63); (8) inhibition of UL97 kinase in viral infections (He, et al., Journal of Virology 1997, 71, 405-11); (9) inhibition of CSF-1R kinase in bone and hematopoetic diseases (Myers, et al., Bioorganic [symbol] Medicinal Chemistry Letters 1997, 7, 421-4), and (10) inhibition of Lck kinase in autoimmune diseases and transplant rejection (Myers, et al., Bioorganic [symbol] Medicinal Chemistry Letters 1997, 7, 417-20).

Inhibitors of certain kinases may also have utility in the treatment of diseases when the kinase is not misregulated, but is nonetheless essential for maintenance of the disease state. In this case, inhibition of the kinase activity would act either as a cure or palliative for these diseases. For example, many viruses, such as human papilloma virus, disrupt the cell cycle and drive cells into the S-phase of the cell cycle (Vousden, FASEB Journal 1993, 7, 872-9). Preventing cells from entering DNA synthesis after viral infection by inhibition of essential S-phase initiating activities such as though kinase inhibition, may disrupt the virus life cycle by preventing virus replication. This same principle may be used to protect normal cells of the body from toxicity of cycle-specific chemotherapeutic agents (Stone, et al., Cancer Research 1996, 56, 3199-202; Kohn, et al., Journal of Cellular Biochemistry 1994, 54, 440-52).

As noted above, GSK3 (glycogen synthase kinase) is identified as a kinase useful in the treatment of type II diabetes. GSK3 inhibits glycogen synthase by direct phosphorylation. Upon insulin activation, GSK3 is inactivated, thereby allowing the activation of glycogen synthase and possibly other insulin-dependent events.

Type II diabetes, otherwise known as Non-Insulin Dependent Diabetes Mellitus (NIDDM), is initially characterized by decreased sensitivity to insulin (insulin resistance) and a compensatory elevation in circulating insulin concentrations. Increased insulin levels are caused by increased secretion from the pancreatic beta cells in an attempt to overcome the insulin resistance. The resulting hyperinsulinemia is associated with a variety of cardiovascular complications.

As insulin resistance worsens, the demand on the pancreatic beta cells steadily increases until the pancreas can no longer provide adequate levels of insulin, thereby resulting in elevated levels of glucose in the blood. Thus, diabetes causes impaired glucose transport into skeletal muscle and increased hepatic glucose production, in addition to inadequate insulin response. The disorders and conditions associated with hyperglycemia and hyperlipidemia include cardiovascular disease, renal failure, and blindness.

GSK3 inhibition stimulates insulin-dependent processes and is consequently useful in the treatment of diseases and conditions, such as type II diabetes, that are mediated by GSK3 activity, or, more specifically, characterized by a need for the inhibition of GSK3.

For example, Klein et al., PNAS 93:8455-9 (1996) report that lithium ion inhibits GSK3 activity. Lithium has been reported to have anti-diabetic effects such as reduction of plasma glucose levels, increased glycogen uptake, potentiation of insulin, and stimulation of glycogen synthesis in skin, muscle, and fat cells. Lithium, however, effects molecular targets other than GSK3, and is, therefore, not a widely accepted therapy for diabetics.

GSK3 is a proline-directed serine/threonine kinase. Other examples of GSK3 mediated diseases or conditions include, without limitation, obesity, various CNS disorders such as Alzheimer's Disease, bipolar disorder, and schizophrenia, neurotraumatic injuries such as acute stroke, immune potentiation, baldness or hair loss, atherosclerotic cardiovascular disease, hypertension, polycystic ovary syndrome, ischemia, brain trauma or injury, immunodeficiency, and cancer. See, for example, published PCT application WO 00/38675, the background of which is herein incorporated by reference.

In addition other tyrosine kinases, such as TIE, also are implicated by the compounds of the present invention. The acronym TIE represents “tyrosine kinase containing Ig and EGF homology domains.” TIE is used to identify a class of receptor tyrosine kinases, which are exclusively expressed in vascular endothelial cells and early hemopoietic cells. Angiopoieten 1 (Ang1), a ligand for the endothelium-specific receptor tyrosine kinase TIE-2, is an angiogenic factor. See, Davis et al, Cell, 1996, 87:1161-1169; Partanen et al., Mol. Cell Biol, 12:1698-1707 (1992); U.S. Pat. Nos. 5,521,073; 5,879,672; 5,877,020; and 6,030,831. Ang1 and its receptor TIE-2 function in the later stages of vascular development, i.e., during vascular remodeling (remodeling refers to formation of a vascular lumen) and maturation. See, Yancopoulos et al., Cell, 1998, 93:661-664; Peters, K. G., Circ. Res., 1998, 83(3):342-3; Suri et al., Cell, 87, 1171-1180 (1996). Consequently, inhibition of TIE-2 would be expected to disrupt remodeling and maturation of new vasculature initiated by angiogenesis thereby disrupting the angiogenic process. Thus, inhibition of TIE-2 should prevent tumor angiogenesis and serve to retard or eradicate tumor growth. Accordingly, a treatment for cancer or other disorders associated with inappropriate angiogenesis could be provided.

As used herein, angiogenesis is defined as involving (i) activation of endothelial cells; (ii) increased vascular permeability; (iii) subsequent dissolution of the basement membrane and extravisation of plasma components leading to formation of a provisional fibrin gel extracellular matrix; (iv) proliferation and mobilization of endothelial cells; (v) reorganization of mobilized endothelial cells to form functional capillaries; (vi) capillary loop formation; and (vii) deposition of basement membrane and recruitment of perivascular cells to newly formed vessels. Normal angiogenesis is activated during tissue growth, from embryonic development through maturity, and then enters a period of relative quiescence during adulthood. Normal angiogensesis is also activated during wound healing, and at certain stages of the female reproductive cycle. Inappropriate angiogenesis has been associated with several disease states including various retinopathies; ischemic disease; atherosclerosis; chronic inflammatory disorders; and cancer. The role of angiogenesis in disease states is discussed in Fan et al., Trends in Pharmacol Sci. 16:54-66; Shawver et al., DDT Vol. 2, No. 2 Feb. 1997; Folkmann, 1995, Nature Medicine, 1:27-31.

For example, in cancer, the growth of solid tumors has been shown to be angiogenesis dependent. See Folkmann, J., J. Nat'l. Cancer Inst., 1990, 82, 4-6. Consequently, the targeting of pro-angiogenic pathways in cancer treatment is a strategy being widely pursued in order to provide new therapeutics in these areas of great, unmet medical need. The role of tyrosine kinases involved in angiogenesis and in the vascularization of solid tumors may prove useful in the creation of effective mediacaments.

Thus, the compounds of the present invention are believed useful is a variety of disease states, each of which may be characterized as mediated by inhibition or antagonism of protein kinases.

### SUMMARY OF THE INVENTION

The present invention provides a method for the treatment or prophylaxis of a disease or condition, said disease or condition characterized by misregulation of a protein kinase, comprising administering of a compound of Formula (I): 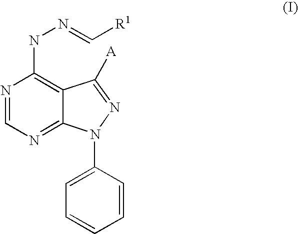
including salts, solvates, and pharmaceutically acceptable derivatives thereof, wherein A is H, alkyl, or aryl; R¹ is D¹, D², D³, D⁴, or D⁵, wherein D¹ is 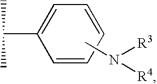
and R³ and R⁴ are each independently H, alkyl, alkylsulfonyl, or —C(O)—(CH₂)ₓ—R⁵, where R⁵ is alkyl, acyl, alkoxy, —(O)—(CH₂)ₓ—(O)-alkyl, or —NR⁶R⁷, where R⁶ and R⁷ are each independently H or alkyl, or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen, or R³ and R⁴ combine to form a 5- or 6-membered ring, optionally containing one or more additional heteroatoms, optionally containing one or more degrees of unsaturation, and optionally substituted one or more times with alkyl, hydroxy, carboxy, alkoxy, acyl, or halogen;
wherein D² is 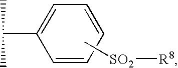
and R⁸ is alkyl, or —NR⁹R¹⁰, where R⁹ and R¹⁰ are each independently selected from H, alkyl, or —(CH₂)ₓ—NR⁶R⁷, where R⁶ and R⁷ are each independently H or alkyl, or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen;
wherein D³ is 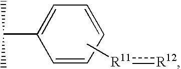
and the dashed line represents an optional double bond; when R¹¹ is —(CH₂)ₓ, the optional dashed double bond does not exist, and R¹² is alkylsulfonyl or —NR¹³R¹⁴, where R¹³ and R¹⁴ are each independently selected from H, alkyl, —(CH₂)ₓ—R¹⁷, where R¹⁷ is alkoxy or —NR¹⁵R¹⁶, where R¹⁵ and R¹⁶ are each independently H or alkyl, or R¹³ and R¹⁴ combine to form a 5- or 6-membered ring, optionally containing one or more additional heteroatoms, optionally containing one or more degrees of unsaturation, and optionally substituted one or more times with alkyl or —(CH₂)ₓ—OH; when R¹¹ is —(CH)—, the optional dashed double bond exists, and R¹² is —(CH)—C(O)—OH;
wherein D⁴ is 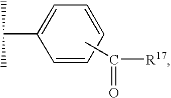
and R¹⁷ is hydroxy, alkoxy, or —NR¹⁸R¹⁹, where R¹⁸ and R¹⁹ are each independently selected from H, alkyl, —(CH₂)ₓ—R²⁰, where R²⁰ is alkylsulfonyl, hydroxy, aryl said aryl optionally substituted with hydroxy or alkoxy, heteroaryl, or —NR²¹R²², where R²¹ and R²² are each independently selected from H, acyl, alkyl, or R²¹ and R²² combine to form a 5- or 6-membered ring, optionally containing one or more additional heteroatoms, optionally containing one or more degrees of unsaturation, and optionally substituted with alkyl or —(CH₂)ₓ—OH; or R¹⁸ and R¹⁹ combine to form a 5- or 6-membered ring, optionally containing one or more additional heteroatoms, optionally containing one or more degrees of unsaturation, and optionally substituted with —(CH₂)ₓ—R²³, where R²³ is alkoxy, hydroxy, —C(O)—R²⁴, where R²⁴ is a 5- or 6-membered ring optionally containing one or more heteroatoms and optionally containing one or more degrees of unsaturation, or —NR²⁵R²⁶, where R²⁵ and R²⁶ are each independently H or alkyl;
wherein D⁵ is a 5- or 6-membered ring, optionally containing one or more heteroatoms, optionally containing one or more degrees of unsaturation, optionally fused with an additional 5- or 6-membered ring that optionally contains one or more heteroatoms and optionally contains one or more degrees of unsaturation, wherein the ring or fused ring system may be optionally substituted one or more times with halogen, alkyl, haloalkyl, alkylsulfonyl, alkylthio, hydroxy, alkoxy, oxo, sulfonyl, sulfate ion, nitro, cyano, carboxy, alkoxyearbonyl, aryl where said aryl may be optionally substituted with sulfamoyl, heteroaryl where said heteroaryl may be optionally substituted with alkyl, or —NR²⁷R²⁸, where R²⁷ and R²⁸ are each independently H, alkyl, acyl, alkoxy, alkoxycarbonyl, carboxy, or —(CH₂)ₓ—NR²⁹R³⁰, where R²⁹ and R³⁰ are each independently selected from H and alkyl, or R²⁷ and R²⁸ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen, or —(O)_{y}—(CH₂)ₓ—R³¹, where R³¹ is hydroxy, alkoxy, haloalkyl, aryl optionally substituted with halogen, or —NR²⁷R²⁸, where R²⁷ and R²⁸ are as defined above; provided that if D⁵ is phenyl, said phenyl must be substituted wherein for each occurrence, x independently is 0, 1, 2, or 3; and wherein for each occurrence, y independently is 0 or 1.

Preferably, in an embodiment of the present invention R¹ is D⁵, where, more preferably, D⁵ is pyridyl substituted one or more times with alkoxy, halogen, —NR²⁷R²⁸, where R²⁷ is H or alkyl and R²⁸ is H, alkyl, acyl, alkoxycarbonyl, or —(CH₂)ₓ—N²⁹R³⁰, where x is 2 and R²⁹ and R³⁰ are each alkyl, or —(O)_{y}—(CH)ₓ—R³¹, where y is 1, x is 2, and R³¹ is —NR²⁷R²⁸, where R²⁷ and R²⁸ are each alkyl.

In another embodiment, more preferably D⁵ is quinolinyl.

In another embodiment, more preferably D⁵ is piperidinyl optionally substituted with alkoxycarbonyl.

In another embodiment R¹ is D² and R⁸ is —NR⁹R¹⁰, where R⁹ is H, and R¹⁰ is H or —(CH₂)ₓ—NR⁶R⁷, where x is 2 or 3, and R⁶ and R⁷ are each alkyl or R⁶ and R⁷ combine to form morpholinyl or pyrrolidinyl.

In another embodiment R¹ is D⁴; and R¹⁷ is hydroxy or —NR¹⁸R¹⁹, where R¹⁸ is H or alkyl, and R¹⁹ is —(CH₂)ₓ—R²⁰, where x is 2 or 3, and R²⁰ is alkylsulfonyl, pyridyl, imidazolyl, or —NR²¹R²², where R²¹ and R²² are each H or alkyl, or R²¹ and R²² combine to form piperidinyl, pyrrolidinyl, morpholinyl, or piperazinyl, each optionally substituted with alkyl, or R¹⁸ and R¹⁹ combine to form piperizinyl optionally substituted with —(CH₂)ₓ—R²³, where x is 2 and R²³ is alkoxy or —NR²⁵R²⁶, where R²⁵ and R²⁶ are each alkyl.

In another embodiment R¹ is D⁵, where, more preferably, D⁵ is phenyl substituted one or more times with alkoxycarbonyl, hydroxy, halogen, alkoxy, carboxy, or —(O)_{y}—(CH₂)ₓ—R³¹, where y is 0 or 1, x is 1 or 2, and R³¹ is hydroxy.

Preferably the kinase is a serine/threosine kinase. More preferably the kinase is GSK3.

In another embodiment, preferably the kinase is a tyrosine kinase. More preferably, the kinase is TIE2.

One aspect of the invention provides the method of the present invention where the disease or condition is type 2 diabetes, hyperlipidemia, obesity, CNS disorders, neurotraumatic injuries, immune potentiation, baldness or hair loss, atherosclerotic cardiovascular disease, hypertension, polycystic ovary syndrome, ischemia, immunodeficiency, or cancer. One embodiment provides for the disease or condition to be type 2 diabetes with the method of the present invention further preferably including administering at least one additional anti-diabetic agent.

Another aspect of the present invention includes the use of a compound as herein described in the preparation of a medicament for use in the treatment of a disease or condition wherein said disease or condition is characterized by misregulation of one or more protein kinase. In one embodiment the kinase is a serine/threosine kinase. More preferably, the kinase is GSK3. In another embodiment, the kinase is a tyrosine kinase. More preferably, the kinase is TIE2.

Preferably, such use is provided when the disease or condition is type 2 diabetes, hyperlipidemia, obesity, CNS disorders, neurotraumatic injuries, immune potentiation, baldness or hair loss, atherosclerotic cardiovascular disease, hypertension, polycystic ovary syndrome, ischemia, immunodeficiency, or cancer. More preferably, the disorder is type 2 diabetes and includes the administration at least one additional anti-diabetic agent.

### DETAILED DESCRIPTION OF THE PREFERRED EMBODIMENT

The term “alkyl” refers to a straight or branched chain hydrocarbon that may be optionally substituted, with multiple degrees of substitution being allowed. Examples of “alkyl” include, but are not limited to methyl, ethyl, n-propyl, isopropyl, n-butyl, t-butyl, n-pentyl, isobutyl, isopropyl, and the like. The phrase “Cₓ-C_{y}alkyl” refers to an alkyl group, as defined above, containing the specified number of carbon atoms.

The term “alkylene” refers to a straight or branched chain unsaturated aliphatic hydrocarbon radical that may be optionally substituted, with multiple degrees of substitution being allowed. Examples of “alkylene” include, but are not limited to methylene, ethylene, n-propylene, n-butylene, and the like.

The term “aryl” refers to an optionally substituted benzene ring or to an optionally substituted benzene ring system fused to one or more optionally substituted benzene rings to form, for example, anthracene, phenanthrene, or naphthalene ring systems. Examples of “aryl” groups include, but are not limited to phenyl, 2-naphthyl, 1-naphthyl, biphenyl, as well as substituted derivatives thereof. The term “aralkyl” further refers to groups of —RₐR_{b}, where Rₐ is an alkylene as defined herein and R_{b} is an aryl as defined herein. Exemplary “aralkyl” groups include C₁₋₆alkylene-aryl, such as benzyl.

The term “heteroaryl” refers to a monocyclic aromatic ring system, or to a fused bicyclic aromatic ring system comprising two or more aromatic rings. These heteroaryl rings contain one or more nitrogen, sulfur, and/or oxygen atoms, where N-oxides and sulfur oxides and dioxides are permissible heteroatom substitutions and may be optionally substituted, with multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof. The term “heteroaralkyl” further refers to groups of —RₐR_{b}, where Rₐ is an alkylene as defined herein and R_{b} is a heteroaryl as defined herein.

As used herein, the term “acyl” refers to the group —C(O)Rₐ, where Rₐ is H, alkyl, or aryl. Non-limiting examples of “acyl” groups include formyl, acetyl, benzoyl, and the like.

The term “alkoxy” refers to the group —ORₐ, where Rₐ is alkyl as defined above. Non-limiting examples of “alkoxy” groups include methoxy, ethoxy, and the like.

As used herein, the term “oxo” refers to the group ═O.

As used herein, the term “hydroxy” refers to the group —OH.

As used herein, the term “carboxy” refers to the group —COOH.

The term “halogen” refers to fluorine, chlorine, bromine, or iodine.

The term “haloalkyl” refers to an alkyl group, as defined herein, that is substituted with at least one halogen. Non-limiting examples of “haloalkyl” groups include methyl, ethyl, propyl, isopropyl, n-butyl, and t-butyl substituted independently with one or more halogens, e.g., fluoro, chloro, bromo, and/or iodo. The term “haloalkyl” should be interpreted to include such substituents as perfluoroalkyl and the like.

The term “haloalkoxy” refers to the group —ORₐ, where Rₐ is haloalkyl as defined above.

As used herein, the term “sulfonyl” shall refer to the group —S(O)₂—.

As used herein, the term “alkylsulfonyl” refers to the group —S(O)₂Rₐ, where Rₐ is alkyl as defined above.

As used herein, the term “alkylthio” refers to the group —SRₐ, where Rₐ is alkyl as defined above.

As used herein, the term “sulfamoyl” refers to a group —SO₂—NH₂.

As used herein, the term “carbamoyl” refers to the group —C(O)NH₂.

As used herein, the term “carboxamide” refers to the group —C(O)N(Rₐ)₂, where Rₐ is alkyl or aryl as defined herein.

As used herein, the term “alkoxycarbonyl” refers to the group —C(O)ORₐ, where Rₐ is alkyl or aryl as defined herein.

The compounds of the present invention may have the ability to crystallize in more than one form, a characteristic known as polymorphism. Such polymorphic forms (“polymorphs”) are within the scope of the present invention. Polymorphism generally can occur as a response to changes in temperature or pressure, or both, and can also result from variations in the crystallization process. Polymorphs can be distinguished by various physical characteristics that are known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers, or enantiomerically or diastereomerically enriched mixtures. Also included within the scope of the invention are the individual isomers of the compounds, as well as any wholly or partially equilibrated mixtures thereof. The present invention also covers the individual isomers of the compounds represented by the formulas above as mixtures with isomers thereof in which one or more chiral centers are inverted.

As noted above, the present invention includes salts, solvates, and pharmaceutically functional derivatives of the compounds of the present invention. Salts include addition salts, metal salts, or optionally alkylated ammonium salts. Examples of such salts include hydrochloric, hydrobromic, hydroiodic, phosphoric, sulfuric, trifluoroacetic, trichloroacetic, oxalic, maleic, pyruvic, malonic, succinic, citric, mandelic, benzoic, cinnamic, methane sulphonic, ethane sulphonic, picric, and the like. Further salts include lithium, sodium, potassium, magnesium, and the like. Reference is also made to Journal of Pharmaceutical Science, 1997, 66, 2, incorporated herein by reference, as relevant to salts.

As used herein, the term “solvate” refers to a complex of variable stoichiometry formed by a solute or a salt or pharmaceutically functional derivative thereof and a solvent. Such solvents for the purpose of the invention should not interfere with the biological activity of the solute. Examples of solvents include, but are not limited to water, methanol, ethanol, and acetic acid. Preferably the solvent used is a pharmaceutically acceptable solvent. Examples of pharmaceutically acceptable solvents include water, ethanol, and acetic acid.

The term “pharmaceutically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are recognizable to those skilled in the art, without undue experimentation. Nevertheless reference is made to the teaching of Burger's Medicinal Chemistry and Drug Discovery, 5^{th} Edition, Vol 1: Principles and Practice, which is incorporated herein by reference to the extent of teaching pharmaceutically functional derivatives.

While it is possible that compounds of the present invention may be administered as the raw chemical, preferably the compounds of the present invention are presented as an active ingredient within a pharmaceutical formulation, as are known in the art. Accordingly, the present invention further includes a pharmaceutical formulation comprising a compound of the present invention, or salt, solvate, or functional derivative thereof together with one or more pharmaceutically acceptable carriers. Optionally, other therapeutic and/or prophylactic ingredients may be included in the pharmaceutical formulation. For example, the compounds of the present invention may be combined with other agents, such as, without limitation, one or more other anti-diabetic agent such as insulin, alpha glucosidase inhibitors, biguanides, insulin secretagogues such as sulphonylureas, insulin senstizers such as thiazolidinediones, and/or dipeptidyl peptidase inhibitors.

Formulations of the present invention include those especially formulated for oral, buccal, parental, transdermal, inhalation, intranasal, transmucosal, implant, or rectal administration. Among the variety of administrations, oral administration typically is preferred. For oral administration tablets, capsules, and caplets may contain conventional excipients such as binding agents, fillers, lubricants, disintegrants, and/or wetting agents. Non-limiting examples of binding agents include syrup, acacia, gelatin, sorbitol, tragacanth, mucilage of starch, or polyvinylpyrrolidone (PVP). Non-limiting examples of fillers include, for example, lactose, sugar, microcrystalline cellulose, maize-starch, calcium phosphate or sorbitol. Non-limiting examples of lubricants include, for example, magnesium sterate, stearic acid, talc, polyethylene glycol or silica. Non-limiting examples of disintegrants include, for example, potato starch or sodium starch glycollate. A non-limiting example of a wetting agent includes sodium lauryl sulfate. The tablets additionally may be coated according to methods known in the art.

Alternatively, the compounds of the present invention may be incorporated into oral liquid preparations such as aqueous or oily suspensions, solutions, emulsions, syrups, or elixirs. Moreover, formulations containing these compounds may be presented as a dry product for constitution with water or other suitable vehicle before use. Liquid preparations may contain conventional additives. Non-limiting examples of such additives include suspending agents such as sorbitol syrup, methyl cellulose, glucose/sugar syrup, gelatin, hydroxyethylcellulose, carboxymethyl cellulose, aluminum sterate gel or hydrogenated edible fats. Additionally, emulsifying agents such as lecithin, sorbitan mono-oleate or acacia; non-aqueous vehicles (which may include edible oils) such as almond oil, fractionated coconut oil, oily esters, propylene glycol or ethyl alcohol my be included. Further, preservatives such as methyl or propyl p-hydroxybenzoates or sorbic acid, may be incorporated into the preparation. Such preparations may also be formulated as suppositories, for example, containing conventional suppository bases such as cocoa butter or other glycerides.

Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, for example, sterile, pyrogen-free water, before use.

The formulations according to the invention may also be formulated as a depot preparation. Such long acting formulations may be administered by implantation, for example, subcutaneously or intramuscularly, or by intramuscular injection. Accordingly, the compounds of the invention may be formulated with suitable polymeric or hydrophobic materials, such as an emulsion in an acceptable oil, ion exchange resins, or as sparingly soluble derivatives, such as a sparingly soluble salt.

Pharmaceutical formulations may be presented in unit dose forms containing a predetermined amount of active ingredient per unit dose. Such a unit may contain certain amounts of a compound of the present invention depending on the condition being treated, the route of administration, and the age, weight and condition of the patient. Preferred unit dosage formulations are those containing a predetermined dose, such as a daily dose, or an appropriate fraction thereof, of an active ingredient. Such pharmaceutical formulations may be prepared by any of the methods well known in the pharmacy art.

As used herein, the term “effective amount” means that amount of a drug or pharmaceutical agent that will elicit the biological or medical response of a tissue, system, animal, or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

A “therapeutically effective amount” of a compound of the present invention will depend upon a number of factors including, for example, the age and weight of the animal, the precise condition requiring treatment and its severity, the nature of the formulation, and the route of administration. Therapeutic effectiveness ultimately will be at the discretion of the attendant physician or veterinarian. An effective amount of a salt or solvate, or pharmaceutically functional derivative thereof, may be determined as a proportion of the effective amount of a compound of the present invention per se.

### EXPERIMENTALS

The following examples illustrate aspects of this invention, but should not be construed as limitations. Unless otherwise noted, all starting materials were obtained from commercial suppliers or obtained through synthetic methods known to those skilled in the art. As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); RT (room temperature);
min (minutes); h (hours);
mp (melting point); TLC (thin layer chromatography);
Tr (retention time); RP (reverse phase);
MeOH (methanol); l-PrOH (isopropanol);
TEA (triethylamine); TFA (trifluoroacetic acid);
TFAA (trifluoroacetic anhydride); THF (tetrahydrofuran);
DMSO (dimethylsulfoxide); EtOAc (ethyl acetate);
DCE (dichloroethane); DMF (N,N-dimethylformamide);
HOAc (acetic acid); EDC (ethylcarbodiimide hydrochloride);
mCPBA (meta-chloroperbenzoic acid;
BOC (tert-butyloxycarbonyl); CBZ (benzyloxycarbonyl);
DCC (dicyclohexylcarbodiimide); Me (methyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine);
HPLC (high pressure liquid chromatography);
Et (ethyl); tBu (tert-butyl).

All references to ether are to diethyl ether; brine refers to a saturated aqueous solution of NaCl. Unless otherwise indicated, all temperatures are expressed in ° C. (degrees Centigrade). All reactions were conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APliii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a MeI-Temp II apparatus and are uncorrected.

IUPAC names are included to further identify particular compounds of the present invention. The IUPAC names stated herein should in no way limit the scope of the present invention. 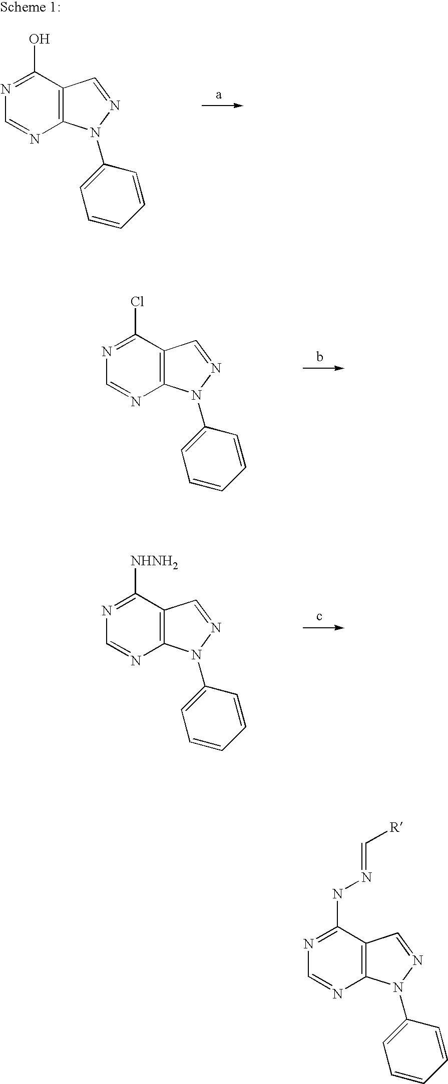
a: phosphorus oxychloride; b: hydrazine hydrate (6 eq), ethanol; c: appropriate aldehyde (1 eq), pyrrolidine (cat.), ethanol. 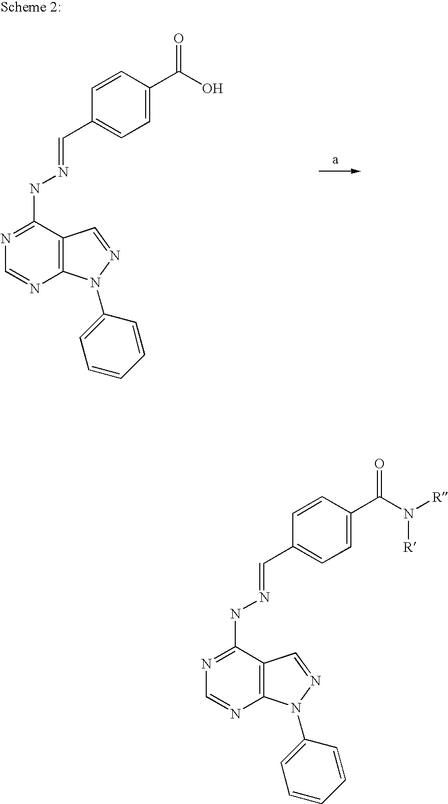
a: appropriate amine (1.5 eq), diethylcyanophosphonate (2 eq), triethylamine (3 eq), DMF 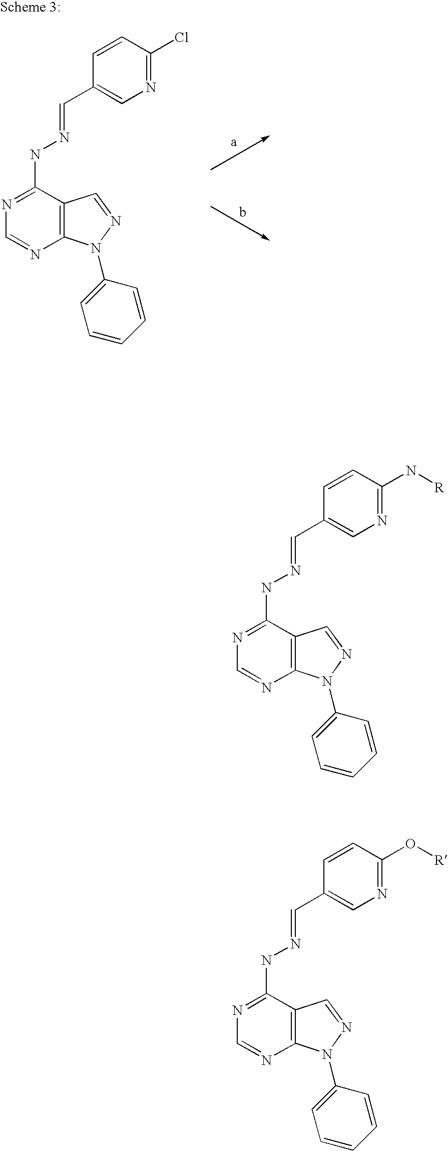
a: appropriate amine, diisopropylethylamine.b: i:Sodium hydride (12 eq), appropriate alcohol (18 eq), THF ii: DMSO

### EXAMPLES

### Example 1

### Isonicotinaldehyde (1-phenyl-1H-pyrazolo[3,4-d]pyrimidin-4-yl)hydrazone

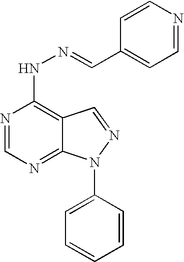

To a stirred solution of 4-hydrazino-1-phenyl-1H-pyrazolo[3,4-d]pyrimidine (250 mg, 1.1 mmol) in ethanol (10 ml) was added isonicotinaldehyde (0.2 ml, 2.2 mmol), and pyrrolidine (1 drop). The reaction mixture was refluxed for 2 hours. The cooled solution was filtered to collect pure product as a yellow solid (220 mg, 63% yield).

¹H NMR (300 MHz, DMSO) δ 12.51 (s, 1H), 8.69 (d, 3H), 8.55 (s, 1H), 8.29 (s, 1H), 8.22 (d, 2H), 7.80 (d, 2H), 7.59 (t, 2H), 7.38 (t, 1H); AP-MS m/z 316 (MH).

### Example 2

### Nicotinaldehyde (1-phenyl-1H-pyrazolo[3,4-d]pyrimidin-4-yl)hydrazone

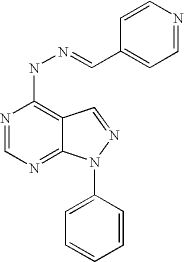

Prepared from 4-hydrazino-1-phenyl-1H-pyrazolo[3,4-d]pyrimidine and nicotinaldehyde using the general procedure for isonicotinaldehyde (1-phenyl-1H-pyrazolo[3,4-d]pyrimidin-4-yl)hydrazone.

¹H NMR (300 MHz, DMSO) δ 12.51 (s, 1H), 9.08 (s, 1H), 8.74 (d, 1H), 8.72 (s, 1H), 8.62 (d, 1H), 8.53 (s, 1H), 8.39 (s, 1H), 8.20 (d, 2H), 7.76 (dd, 1H), 7.57 (t, 2H), 7.37 (t, 1H). ES-MS m/z 316 (MH⁺).

### Example 3

### Methyl 3-{(E)-[(1-phenyl-1H-pyrazolo[3,4-d]pyrimidin-4-yl)hydrazono]methyl}benzoate

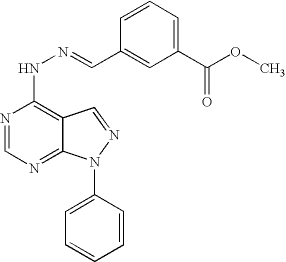

Prepared from 4-hydrazino-1-phenyl-1H-pyrazolo[3,4-d]pyrimidine and methyl 4-formylbenzoate using the general procedure for isonicotinaldehyde (1-phenyl-1H-pyrazolo[3,4-d]pyrimidin-4-yl)hydrazone.

¹H NMR (400 MHz, DMSO) δ 12.40 (s, 1H), 8.63 (s, 1H), 8.50 (s, 1H), 8.38 (s, 1H), 8.29 (s, 1H), 8.25-8.13 (m, 3H), 8.01 (d, 1H), 7.66 (t, 1H), 7.57 (t, 2H), 7.37 (t, 1H), 3.90 (s, 3H); ES-MS m/z 372 (MH⁺).

### Example 4

### Quinoline-3-carbaldehyde (1-phenyl-1H-pyrazolo[3,4-d]pyrimidin-4-yl)hydrazone

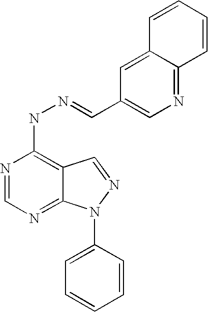

Prepared from 4-hydrazino-1-phenyl-1H-pyrazolo[3,4-d]pyrimidine and quinoline-3-carbaldehyde using the general procedure for isonicotinaldehyde (1-phenyl-1H-pyrazolo[3,4-d]pyrimidin-4-yl)hydrazone.

¹H NMR (300 MHz, DMSO) δ 9.43 (s, 1H), 8.83 (s, 1H), 8.77 (s, 1H), 8.55 (s, 2H), 8.27 (d, 2H), 8.19 (d, 1H), 8.10 (d, 1H), 7.85 (t, 1H), 7.71 (t, 1H), 7.62 (t, 2H), 7.41 (t, 1H). ES-MS m/z 366 (MH⁺).

### Example 5

### 3-(2-Hydroxyethoxy)benzaldehyde (1-phenyl-1H-pyrazolo[3,4-d]pyrimidin-4-yl)hydrazone

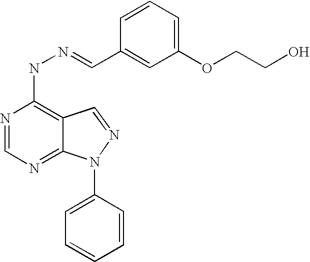

Prepared from 4-hydrazino-1-phenyl-1H-pyrazolo[3,4-d]pyrimidine and 3-(2-hydroxyethoxy)benzaldehyde using the general procedure for isonicotinaldehyde (1-phenyl-1H-pyrazolo[3,4-d]pyrimidin-4-yl)hydrazone.

¹H NMR (400 MHz, DMSO) δ 8.58 (s, 1H), 8.47 (s, 1H), 8.26 (s, 1H), 8.20 (d, 2H), 7.55 (t, 2H), 7.38 (m, 2H), 7.35 (t, 1H), 7.31 (s, 1H), 7.02 (m, 2H), 4.06 (t, 2H), 3.74 (m, 2H). ES-MS m/z 375 (MH⁺).

### Example 6

### 3-Hydroxybenzaldehyde (1-phenyl-1H-pyrazolo[3,4-d]pyrimidin-4-yl)hydrazone

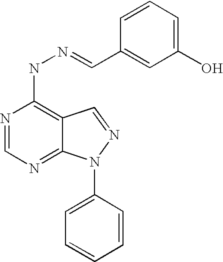

Prepared from 4-hydrazino-1-phenyl-1H-pyrazolo[3,4-d]pyrimidine and 3-hydroxybenzaldehyde using the general procedure for isonicotinaldehyde (1-phenyl-1H-pyrazolo[3,4-d]pyrimidin-4-yl)hydrazone.

¹H NMR (400 MHz, DMSO) δ 12.21 (s, 1H), 9.71 (s, 1H), 8.63 (s, 1H), 8.55 (s, 1H), 8.19-8.47 (m, 3H), 7.55 (t, 2H), 8.35 (s, 2H), 7.27 (t, 1H), 7.11 (d, 1H), 6.82 (d, 1H). ES-MS m/z 331 (MH⁺).

### Example 7

### 3-Hydroxy-4,5-dimethoxybenzaldehyde (1-phenyl-1H-pyrazolo[3,4-d]pyrimidin-4-yl)hydrazone

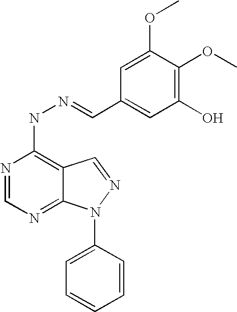

Prepared from 4-hydrazino-1-phenyl-1H-pyrazolo[3,4-d]pyrimidine and 3-hydroxy-4,5-dimethoxybenzaldehyde using the general procedure for isonicotinaldehyde (1-phenyl-1H-pyrazolo[3,4-d]pyrimidin-4-yl)hydrazone.

¹H NMR (400 MHz, DMSO) δ 12.06 (s, 1H), 9.58 (s, 1H), 8.61 (s, 1H), 8.45 (s, 1H), 8.20 (d, 2H), 8.11 (s, 1H), 7.55 (t, 2H), 7.34 (t, 1H), 7.09 (s, 1H), 6.81 (s, 1H), 3.83 (s, 3H), 3.70 (s, 3H). ES-MS m/z 395 (MH⁺).

### Example 8

### tert-butyl 4-{(E)-[(1-phenyl-1H-pyrazolo[3,4-d]pyrimidin-4-yl)hydrazono]methyl}piperidine-1-carboxylate

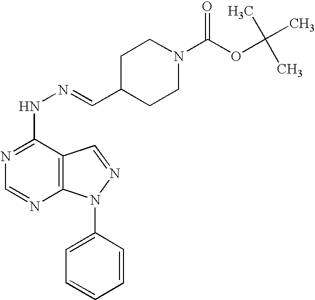

Prepared from 4-hydrazino-1-phenyl-1H-pyrazolo[3,4-d]pyrimidine and tert-butyl 4-formylpiperidine-1-carboxylate using the general procedure for isonicotinaldehyde (1-phenyl-1H-pyrazolo[3,4-d]pyrimidin-4-yl)hydrazone.

¹H NMR (400 MHz, DMSO) δ 11.86 (s, 1H), 8.41 (s, 2H), 8.19 (d, 2H), 7.59-7.51 (m, 3H), 7.34 (t, 1H), 4.01-3.95 (m, 2H), 2.88-2.80 (m, 2H), 2.66-2.51 (m, 1H), 1.95-1.83 (m, 2H), 1.43-1.32 (m, 11H); ES-MS m/z 422 (MH⁺).

### Example 9

### Piperidine-4-carbaldehyde (1-phenyl-1H-pyrazolo[3,4-d]pyrimidin-4-yl)hydrazone trifluoroacetate

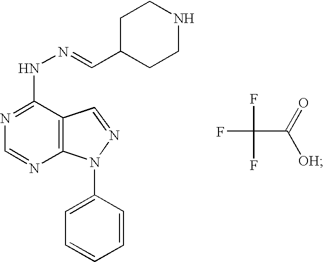

A solution of tert-butyl 4-{(E)-[(1-phenyl-1H-pyrazolo[3,4-d]pyrimidin-4-yl)hydrazono]methyl}piperidine-1-carboxylate (50 mg, 0.119 mmol), dichloromethane (5 ml), and TFA (0.05 ml, 0.594 mmol) was stirred at rt for 16 h. To the solution was added additional TFA (1 ml) and the mixture was heated at 40° C. for 15 min. The resulting mixture was concentrated, washed with chloroform, and collected by filtration to give piperidine-4-carbaldehyde (1-phenyl-1H-pyrazolo[3,4-d]pyrimidin-4-yl)hydrazone trifluoroacetate as an off white solid (45 mg, 93% yield).

¹H NMR (400 MHz, DMSO) δ 11.97 (s, 1H), 8.68-8.56 (m, 1H), 8.51-8.41 (m, 2H), 8.38-8.23 (m, 1H), 8.17 (d, 2H), 7.65 (s, 1H), 7.55 (t, 2H), 7.42-7.28 (m, 1H), 3.46-3.28 (m, 1H), 3.05-2.90 (m, 2H), 2.78-2.62 (m, 1H), 2.16-2.02 (m, 2H), 1.77-1.61 (m, 2H); ES-MS m/z 322 (MH⁺).

### Example 10

### N-(5-{(E)-[2-(1-phenyl-1H-pyrazolo[3,4-d]pyrimidin-4-yl)hydrazono]methyl}pyridin-2-yl)acetamide

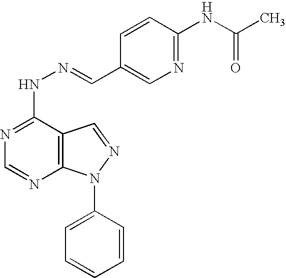

Prepared from 4-hydrazino-1-phenyl-1H-pyrazolo[3,4-d]pyrimidine and N-(5-formylpyridin-2-yl)acetamide using the general procedure for isonicotinaldehyde (1-phenyl-1H-pyrazolo[3,4-d]pyrimidin-4-yl)hydrazone.

¹H NMR (400 MHz, DMSO) δ 12.35 (s, 1H), 10.75 (s, 1H), 8.68 (s, 1H), 8.62 (s, 1H), 8.48 (s, 1H), 8.35 (d, 1H), 8.28 (s, 1H), 8.25-8.15 (m, 3H), 7.56 (t, 2H), 7.36 (t, 1H), 2.12 (s, 3H); ES-MS m/z 373 (MH⁺).

### Example 11

### tert-butyl 5-{(E)-[(1-phenyl-1H-pyrazolo[3,4-d]pyrimidin-4-yl)hydrazono]methyl}pyridin-2-ylcarbamate

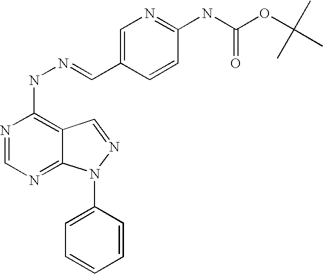

Prepared from 4-hydrazino-1-phenyl-1H-pyrazolo[3,4-d]pyrimidine and tert-butyl 5-formylpyridin-2-ylcarbamate using the general procedure for isonicotinaldehyde (1-phenyl-1H-pyrazolo[3,4-d]pyrimidin-4-yl)hydrazone.

¹H NMR (300 MHz, DMSO) δ 12.28 (s, 1H), 10.06 (s, 1H), 8.67 (d, 1H), 8.56 (s, 1H), 8.47 (s, 1H), 8.29 (d, 1H), 8.26 (s, 1H), 8.21 (d, 2H), 7.93 (d, 1H), 7.56 (t, 2H), 7.35 (t, 1H), 1.48 (s, 9H). ES-MS m/z 431.1 (MH⁺).

### Example 12

### 6-aminonicotinaldehyde (1-phenyl-1H-pyrazolo[3,4-d]pyrimidin-4-yl)hydrazone trifluoroacetate

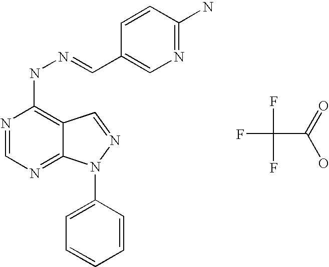

Trifluoroacetic acid (1 mL) was added to a suspension of tert-butyl 5-{(E)-[(1-phenyl-1H-pyrazolo[3,4-d]pyrimidin-4-yl)hydrazono]methyl}pyridin-2-ylcarbamate (0.22 g; 0.48 mmol) in CH₂Cl₂ (5 mL). The mixture was stirred at reflux for 3 h then the solvent was removed to give the title compound (0.21 g) as a yellow solid (99%).

¹H NMR (300 MHz, CD₃OD) δ 8.63-8.44 (m, 3H), 8.23-8.11 (m, 3H), 7.55 (t, 2H), 7.42-7.37 (m, 1H), 7.16-7.08 (m, 2H). ES-MS m/z 329 (MH⁻).

### Example 13

### 6-(dimethylamino)nicotinaldehyde (1-phenyl-1H-pyrazolo[3,4-d]pyrimidin-4-yl)hydrazone

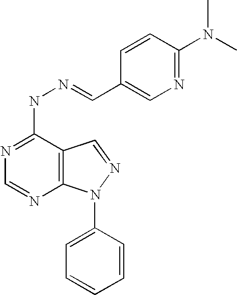

Prepared from 4-hydrazino-1-phenyl-1H-pyrazolo[3,4-d]pyrimidine and 6-(dimethylamino)nicotinaldehyde using the general procedure for isonicotinaldehyde (1-phenyl-1H-pyrazolo[3,4-d]pyrimidin-4-yl)hydrazone.

¹H NMR (300 MHz, DMSO) δ 12.25 (s, 1H), 8.60 (s, 1H), 8.44 (s, 1H), 8.30 (s, 1H), 8.26-8.19 (m, 4H), 7.55 (t, 2H), 7.35 (t, 1H), 6.99 (d, 1H), 3.17 (s, 6H).

### Example 14

### 2-chloroisonicotinaldehyde (1-phenyl-1H-pyrazolo[3,4-d]pyrimidin-4-yl)hydrazone

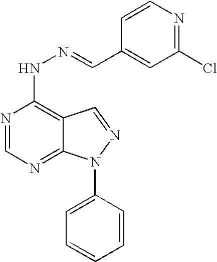

Prepared from 4-hydrazino-1-phenyl-1H-pyrazolo[3,4-d]pyrimidine and 2-chloroisonicotinaldehyde using the general procedure for isonicotinaldehyde (1-phenyl-1H-pyrazolo[3,4-d]pyrimidin-4-yl)hydrazone.

¹H NMR (400 MHz, DMSO) δ 12.64 (s, 1H), 8.68 (s, 1H), 8.55 (s, 1H), 8.48 (d, 1H), 8.26 (s, 1H), 8.20 (d, 2H), 7.89 (d, 1H), 7.83 (s, 1H), 7.57 (t, 2H), 7.37 (t, 1H); ES-MS m/z 350 (MH⁺).

### Example 15

### 2-methoxyisonicotinaldehyde (1-phenyl-1H-pyrazolo[3,4-d]pyrimidin-4-yl)hydrazone

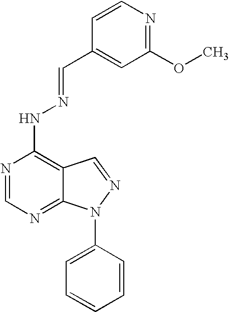

Sodium methoxide (50 mg) was added to a solution of 2-chloroisonicotinaldehyde (1-phenyl-1H-pyrazolo[3,4-d]pyrimidin-4-yl)hydrazone (50 mg, 0.14 mmol) in DMSO (2 ml). The mixture was stirred at 80° C. for 1 h, cooled to RT and water was added. The resulting solid was collected by filtration, washed with water, and air dried to give 2-methoxyisonicotinaldehyde (1-phenyl-1H-pyrazolo[3,4-d]pyrimidin-4-yl)hydrazone as a pure product (18 mg, yield 37%).

¹H NMR (300 MHz, DMSO) δ 12.50 (s, 1H), 8.63 (s, 1H), 8.54 (s, 1H), 8.27-8.18 (m, 4H), 7.58 (d, 2H), 7.50 (d, 1H), 7.38 (t, 1H), 7.10 (s, 1H), 3.89 (s, 3H); ES-MS m/z 346 (MH⁺).

### Example 16

### 6-chloronicotinaldehyde (1-phenyl-1H-pyrazolo[3,4-d]pyrimidin-4-yl)hydrazone

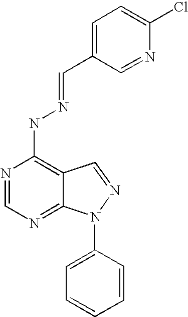

Prepared from 4-hydrazino-1-phenyl-1H-pyrazolo[3,4-d]pyrimidine and 6-chloronicotinaldehyde using the general procedure for isonicotinaldehyde (1-phenyl-1H-pyrazolo[3,4-d]pyrimidin-4-yl)hydrazone.

¹H NMR (300 MHz, DMSO) δ 12.46 (s, 1H), 8.74 (s, 1H), 8.65 (s, 1H), 8.50 (s, 1H), 8.39 (dd, 1H), 8.31 (s, 1H), 8.20 (d, 2H), 7.61-7.54 (m, 3H), 7.35 (t, 1H). ES-MS m/z 348 (MH⁻)

### Example 17

### 6-methoxynicotinaldehyde (1-phenyl-1H-pyrazolo[3,4-d]pyrimidin-4-yl)hydrazone

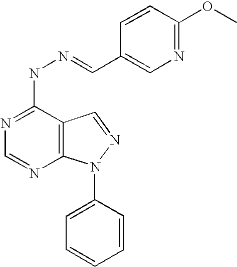

A mixture of 6-chloronicotinaldehyde (1-phenyl-1H-pyrazolo[3,4-d]pyrimidin-4-yl)hydrazone (0.075 g; 0.21 mmol) and sodium methoxide (0.080 g; 1.51 mmol) in DMSO (3 mL) were heated to 105° C. for 1 h. The solution was cooled to RT then water (25 mL) and 1 N HCl (15 mL) were added. The solid was filtered, washed with MeOH (3 mL) then Et₂O (5 mL) and dried to give title compound (71 mg) as a off-white powder (98%).

¹H NMR (300 MHz, DMSO) δ 12.26 (s, 1H), 8.64 (s, 1H), 8.47 (s, 2H), 8.30-8.28 (m, 2H), 8.21 (d, 2H), 7.56 (t, 2H), 7.36 (t, 1H), 6.96 (d, 1H), 3.90 (s, 3H).

### Example 18

### 6-[2-(dimethylamino)ethoxy]nicotinaldehyde (1-phenyl-1H-pyrazolo[3,4-d]pyrimidin-4-yl)hydrazone

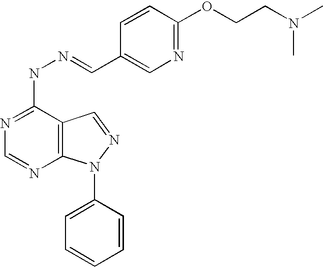

Sodium hydride (0.14 g; 3.50 mmol) was added to a solution of 2-dimethylaminoethanol (0.47 g; 5.28 mmol) in THF (10 mL). After 1 h the solvent was removed under vacuum and DMSO (10 mL) then 6-chloronicotinaldehyde (1-phenyl-1H-pyrazolo[3,4-d]pyrimidin-4-yl)hydrazone (0.10 g; 0.29 mmol) were added. The suspension was heated to 105° C. for 0.5 h then cooled to RT. Water (10 m) was added and the resulting precipitate was filtered, washed with Et₂O (10 mL) and dried to give the title compound (0.11 g; 96%).

¹H NMR (300 MHz, DMSO) δ 12.25 (s, 1H), 8.63 (s, 1H), 8.46 (s, 1H), 8.43 (s, 1H), 8.30-8.27 (m, 2H), 8.21 (d, 2H), 7.56 (t, 2H), 7.35 (t, 1H), 6.92 (d, 1H), 4.39 (t, 2H), 2.62 (t, 2H), 2.20 (s, 6H).

### Example 19

### 6-{[2-(dimethylamino)ethyl]amino}nicotinaldehyde (1-phenyl-1H-pyrazolo[3,4-d]pyrimidin-4-yl)hydrazone hydrochloride

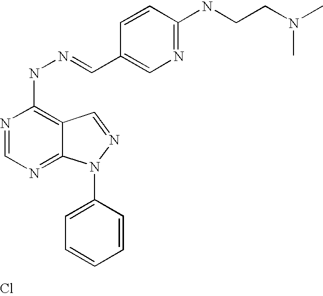

A solution of 6-chloronicotinaldehyde (1-phenyl-1H-pyrazolo[3,4-d]pyrimidin-4-yl)hydrazone (0.15 g; 0.43 mmol), N,N-dimethylethylenediamine (2 mL) and diisopropylethylamine (1 mL) were heated to 105° C. under N₂ for 24 h. The solution was cooled to 0° C. and 3N HCl (3 mL) was added. The resulting precipitate was filtered, dissolved in 3N NaOH, and extracted with ethylacetate. The organic layer was dried over Na₂SO₄, filtered and concentrated. The residue was dissolved in CH₂Cl₂ and 1N HCl in Et₂O was added. The solid was filtered and dried to give title compound (16 mg).

¹H NMR (300 MHz, CDCl₃) δ 8.63 (s, 1H), 8.49 (s, 1H), 8.27 (s, 1H), 8.19 (d, 2H), 7.96 (d, 1H), 7.86 (s, 1H), 7.53 (t, 2H), 7.34 (t, 1H), 6.51 (d, 1H), 5.56 (s, 1H), 3.48-3.43 (m, 2H), 2.58 (t, 2H), 2.29 (s, 6H), 1.27-1.18 (m, 2H). ES-MS m/z 402 (MH⁺).

### Example 20

### 4-{(E)-[(1-phenyl-1H-pyrazolo[3,4-d]pyrimidin-4-yl)hydrazonol]methyl}benzenesulfonamide

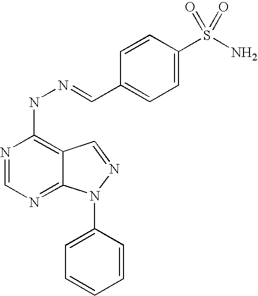

Prepared from 4-hydrazino-1-phenyl-1H-pyrazolo[3,4-d]pyrimidine and 4-formylbenzenesulfonamide using the general procedure for isonicotinaldehyde (1-phenyl-1H-pyrazolo[3,4-d]pyrimidin-4-yl)hydrazone.

¹H NMR (400 MHz, DMSO) δ 8.69 (s, 1H), 8.50 (s, 1H), 8.35 (s, 1H), 8.20 (d, 2H), 8.00 (d, 2H), 7.89 (d, 2H), 7.56 (m, 2H), 7.46 (s, 2H), 7.35 (t, 1H). ES-MS m/z 394 (MH⁺).

### Example 21

### N-(2-morpholin-4-ylethyl)-4-{(E)-[(1-phenyl-1H-pyrazolo[3,4-d]pyrimidin-4-yl)hydrazono]methyl}benzenesulfonamide

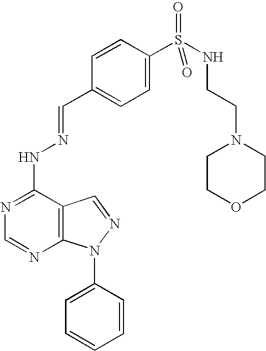

Prepared from 4-hydrazino-1-phenyl-1H-pyrazolo[3,4-d]pyrimidine and 4-formyl-N-(2-morpholin-4-ylethyl)benzenesulfonamide using the general procedure for isonicotinaldehyde (1-phenyl-1H-pyrazolo[3,4-d]pyrimidin-4-yl)hydrazone.

¹H NMR (300 MHz, DMSO) δ 12.66-12.27 (s, 1H), 8.71 (s, 1H), 8.53 (s, 1H), 8.37 (s, 1H), 8.23 (d, 2H), 8.21 (d, 2H), 7.90 (d, 2H), 7.69 (t, 1H), 7.63-7.54 (m, 2H), 7.41-7.36 (m, 1H), 3.52-3.46 (m, 4H), 2.99-2.89 (m, 2H), 2.33-2.20 (m, 6H), 1.75-1.63 (m, 6H); ES-MS m/z 507 (MH⁺).

### Example 22

### N[2-(dimethylamino)ethyl]-4-[{(E)[(1-phenyl-1H-pyrazolo[3,4-d]pyrimidin-4-yl)hydrazono]methyl}benzenesulfonamide hydrochloride

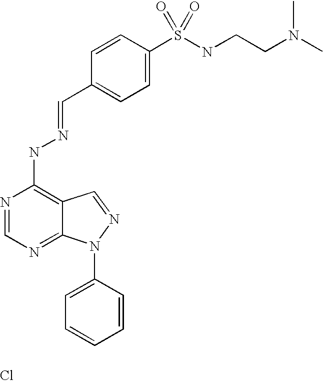

Prepared from 4-hydrazino-1-phenyl-1H-pyrazolo[3,4-d]pyrimidine and N-[2-(dimethylamino)ethyl]-4-formylbenzenesulfonamide using the general procedure for isonicotinaldehyde (1-phenyl-1H-pyrazolo[3,4-d]pyrimidin-4-yl)hydrazone.

¹H NMR (300 MHz, DMSO) δ 12.53 (s, 1H), 10.03 (s, 1H), 8.74 (s, 1H), 8.57 (s, 1H), 8.42 (s, 1H), 8.27-8.22 (m, 3H), 8.12 (d, 2H), 7.97 (d, 2H), 7.62 (t, 2H), 7.41 (t, 1H), 3.45 (m, 4H), 2.80 (s, 6H).

### Example 23

### 4-{(E)-[(1-phenyl-1H-pyrazolo[3,4-d]pyrimidin-4-yl)hydrazono]methyl}-N-(3-pyrrolidin-1-ylpropyl)benzenesulfonamide

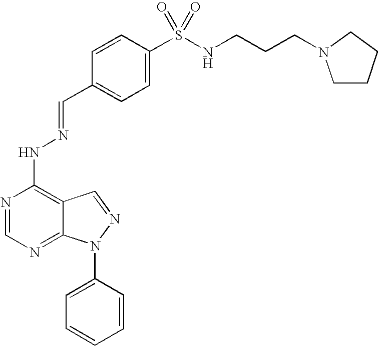

Prepared from 4-hydrazino-1-phenyl-1H-pyrazolo[3,4-d]pyrimidine and 4-formyl-N-(3-pyrrolidin-1-ylpropyl)benzenesulfonamide using the general procedure for isonicotinaldehyde (1-phenyl-1H-pyrazolo[3,4-d]pyrimidin-4-yl)hydrazone.

¹H NMR (300 MHz, DMSO) δ 12.75-12.35 (s br, 1H), 8.74 (s, 1H), 8.57 (s, 1H), 8.57 (s, 1H), 8.41 (s, 1H), 8.25 (d, 2H), 8.08 (d, 2H), 7.93 (d, 2H), 7.62 (t, 2H), 7.41 (t, 1H), 3.40-3.28 (m, 2H) 2.99-2.70 (m, 6H), 1.92-1.63 (m, 6H); ES-MS m/z 505 (MH⁺).

### Example 24

### 4-{(E)-[(1-phenyl-1H-pyrazolo[3,4-d]pyrimidin-4-yl)hydrazono]methyl}benzoic Acid

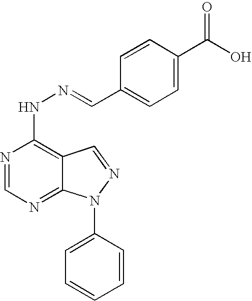

Prepared from 4-hydrazino-1-phenyl-1H-pyrazolo[3,4-d]pyrimidine and 4-formylbenzoic acid using the general procedure for isonicotinaldehyde (1-phenyl-1H-pyrazolo[3,4-d]pyrimidin-4-yl)hydrazone.

¹H NMR (300 MHz, DMSO) δ 12.44 (s br, 1H), 8.07 (s, 1H), 8.53 (s, 1H), 8.37 (s, 1H), 8.23 (d, 2H), 8.06 (d, 2H), 7.96 (d, 2H), 7.59 (t, 2H), 7.38 (t, 1H); AP-MS m/z 359 (MH⁺).

### Example 25

### N-[2-(dimethylamino)ethyl]-4-{(E)-[(1-phenyl-1H-pyrazolo[3,4-d]pyrimidin-4-yl)hydrazono]methyl}benzamide

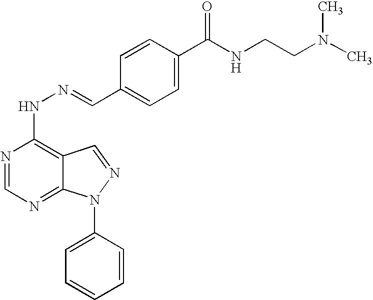

To a solution of 4-{(E)-[(1-phenyl-1H-pyrazolo[3,4-d]pyrimidin-4-yl)hydrazono]methyl}benzoic acid (200 mg, 0.559 mmol) in DMF (15 ml) was added N,N′-dimethylethylenediamine (0.07 ml, 0.645 mmol), diethylcyanophosphonate (0.13 ml, 0.860 mmol), and triethylamine (0.18 ml, 1.29 mmol). The solution was stirred at rt for 3 h, then water and diethyl ether were added. The resulting percipitate was collected by filtration to give pure product (189 mg, yield 79%).

¹H NMR (300 MHz, DMSO) δ 12.36 (s br, 1H), 8.69 (s, 1H), 8.52-8.48 (m, 2H), 8.35 (s, 1H), 8.23 (d, 2H), 7.96-7.90 (m, 4H), 7.58 (t, 2H), 7.38 (t, 1H), 3.41-3.32 (m, 2H), 2.49-2.42 (m, 2H); ES-MS m/z 429 (MH⁺).

### Example 26

### 4-{(E)-[(1-phenyl-1H-pyrazolo[3,4-d]pyrimidin-4-yl)hydrazono]methyl}-N-(3-pyrrolidin-1-ylpropyl)benzamide

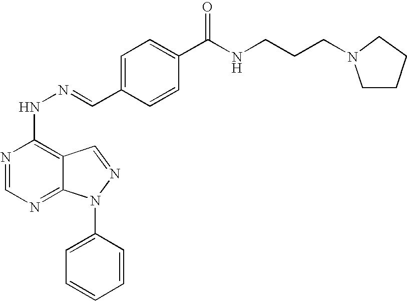

To a solution of 4-{(E)-[(1-phenyl-1H-pyrazolo[3,4-d]pyrimidin-4-yl)hydrazono]methyl}benzoic acid (200 mg, 0.558 mmol) in DMF (10 ml), was added N-(3-aminopropyl)pyrrolidine (0.15 ml, 1.12 mmol), diethylcyanophosphonate (0.17 ml, 1.12 mmol), and triethylamine (0.24 ml, 1.67 mmol). The solution was stirred at rt for 1 h, then water and diethyl ether were added. The resulting percipitate was collected by filtration to give 4-{(E)-[(1-phenyl-1H-pyrazolo[3,4-d]pyrimidin-4-yl)hydrazono]methyl}-N-(3-pyrrolidin-1-ylpropyl)benzamide as a yellow solid (244 mg, yield 93%).

¹H NMR (300 MHz, DMSO) δ 12.30 (s br, 1H), 8.68 (m, 2H), 8.51 (s, 1H), 8.35 (s, 1H), 8.22 (d, 2H), 7.96-7.89 (m, 4H), 7.58 (t, 2H), 7.37 (t, 1H), 3.36-3.28 (m, 2H), 2.53-2.45 (m, 6H), 1.74-1.65 (m, 6H); ES-MS m/z 469 (MH⁺).

### Example 27

### N-(3-Morpholin-4-ylpropyl)-4-[{(E)-[(1-phenyl-1H-pyrazolo[3,4-d]pyrimidin-4-yl)hydrazono]methyl}benzamide

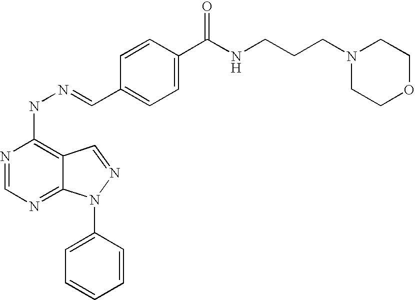

Prepared from 4-{(E)-[(1-phenyl-1H-pyrazolo[3,4-d]pyrimidin-4-yl)hydrazono]methyl}benzoic acid and 3-morpholin-4-ylpropan-1-amine using the method described by 4-{(E)-[(1-phenyl-1H-pyrazolo[3,4-o]pyrimidin-4-yl)hydrazono]methyl}-N-(3-pyrrolidin-1-yl propyl)benzamide.

¹H NMR (300 MHz, DMSO) δ 12.40 (s, 1H), 8.71 (s, 1H), 8.62 (s, 1H), 8.54 (s, 1H), 8.37 (s, 1H), 8.25 (d, 2H), 7.95 (m, 4H), 7.61 (t, 2H), 7.40 (t, 1H), 3.59 (m, 6H), 3.25 (m, 2H), 2.52 (m, 4H), 1.72 (t, 2H). ES-MS m/z 485 (MH⁺).

### Example 28

### 4-{(E)-[(1-phenyl-1H-pyrazolo[3,4-d]pyrimidin-4-yl)hydrazono]methyl}-N-(pyridin-2-ylmethyl)benzamide

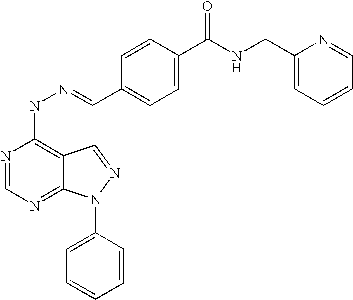

Prepared from 4-{(E)-[(1-phenyl-1H-pyrazolo[3,4-d]pyrimidin-4-yl)hydrazono]methyl}benzoic acid and 1-pyridin-2-ylmethanamine using the method described by 4-[({(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.34 (s, 1H), 9.21 (t, 1H), 8.67 (s, 1H), 8.49 (s, 2H), 8.34 (s, 1H), 8.20 (d, 2H), 8.01 (d, 2H), 7.92 (d, 2H), 7.74 (t, 1H), 7.55 (t, 2H), 7.31-7.37 (m, 2H), 7.24 (t, 1H), 4.56 (d, 2H). ES-MS m/z 449 (MH⁺).

### Example 29

### N[2-(1H-imidazol-4-yl)ethyl]-4-{(E)-[(1-phenyl-1H-pyrazolo[3,4-d]pyrimidin-4-yl)hydrazono]methyl}benzamide

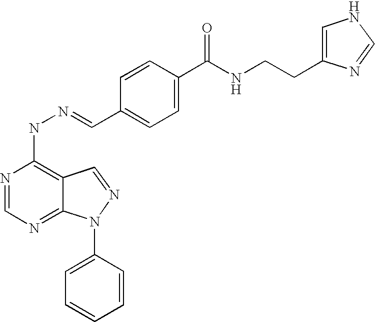

Prepared from 4-{(E)-[(1-phenyl-1H-pyrazolo[3,4-d]pyrimidin-4-yl)hydrazono]methyl}benzoic acid and 2-(1H-imidazol-4-yl)ethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (300 MHz, DMSO) δ 12.38 (s, 1H), 8.79 (t, 1H), 8.65 (s, 1H), 8.48 (s, 1H), 8.32 (s, 1H), 8.18-8.21 (m, 3H), 7.86-7.93 (m, 4H), 7.55 (t, 2H), 7.35 (t, 1H), 7.09 (s, 1H), 3.75 (m, 2H), 2.84 (t, 2H). ES-MS m/z 452 (MH⁺).

### Example 30

### N-Methyl-4-{(E)-[(1-phenyl-1H-pyrazolo[3,4-d]pyrimidin-4-yl)hydrazono]methyl}-N-(2-pyridin-2-ylethyl)benzamide

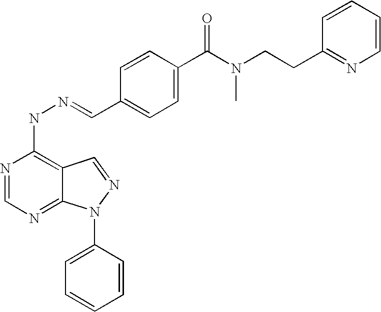

Prepared from 4-{(E)-[(1-phenyl-1H-pyrazolo[3,4-d]pyrimidin-4-yl)hydrazono]methyl}benzoic acid and N-methyl-2-pyridin-2-ylethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.32 (s, 1H), 8.65 (s, 1H), 8.48 (m, 2H), 8.30 (s, 1H), 8.20 (d, 2H), 7.78-7.84 (m, 2H), 7.65-7.71 (m, 1H), 7.55 (t, 2H), 7.41 (d, 1H), 7.35 (t, 1H), 7.14-7.23 (m, 3H), 3.27 (s, 3H), 2.98 (m, 2H), 2.84 (m, 2H). ES-MS m/z 477 (MH⁺).

### Example 31

### N-(2-Aminoethyl)-4-{(E)-[(1-phenyl-1H-pyrazolo[3,4-d]pyrimidin-4-yl)hydrazono]methyl}benzamide

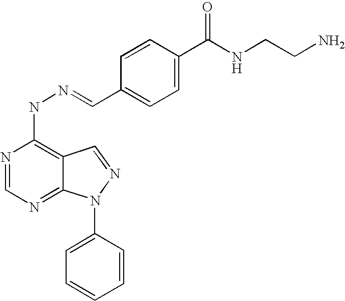

Prepared from 4-{(E)-[(1-phenyl-1H-pyrazolo[3,4-d]pyrimidin-4-yl)hydrazono]methyl}benzoic acid and ethane-1,2-diamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 8.66 (s, 1H), 8.55 (m, 1H), 8.48 (s, 1H), 8.32 (s, 1H), 8.20 (d, 2H), 7.87-7.95 (m, 5H), 7.55 (t, 2H), 7.34 (t, 1H), 3.46 (m, 2H), 2.91 (m, 2H). ES-MS m/z 401 (MH⁺).

### Example 32

### N-(3-aminopropyl)-4-{(E)-[(1-phenyl-1H-pyrazolo[3,4-d]pyrimidin-4-yl)hydrazono]methyl}benzamide

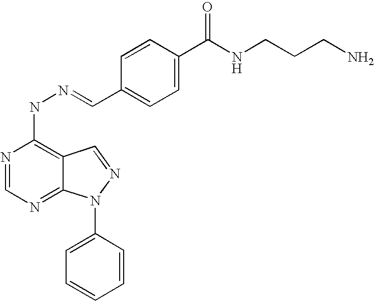

Prepared from 4-{(E)-[(1-phenyl-1H-pyrazolo[3,4-d]pyrimidin-4-yl)hydrazonolmethyl}benzoic acid and propane-1,3-diamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 8.66 (s, 1H), 8.56 (t, 1H), 8.48 (s, 1H), 8.32 (s, 1H), 8.20 (d, 2H), 7.87-7.93 (m, 5H), 7.55 (t, 2H), 7.35 (t, 1H), 3.33 (m, 2H), 2.79 (m, 2H), 1.63 (m, 2H). ES-MS m/z 415 (MH⁺).

### Example 33

### 4-{(E)-[(1-phenyl-1H-pyrazolo[3,4-d]pyrimidin-4-yl)hydrazono]methyl}-N-(2-piperidin-1-ylethyl)benzamide

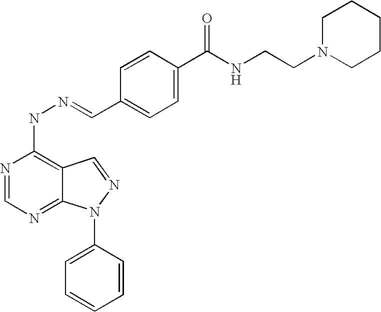

Prepared from 4-{(E)-[(1-phenyl-1H-pyrazolo[3,4-d]pyrimidin-4-yl)hydrazono]methyl}benzoic acid and 2-piperidin-1-ylethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.36 (s, 1H), 8.67 (s, 1H), 8.49 (m, 2H), 8.32 (s, 1H), 8.20 (d, 2H), 7.90 (m, 4H), 7.56 (t, 2H), 7.35 (t, 1H), 3.23 (m, 6H), 2.35 (m, 2H), 1.46 (m, 4H), 1.34 (m, 2H). ES-MS m/z 469 (MH⁺).

### Example 34

### 4-{(E)-[(1-phenyl-1H-pyrazolo[3,4-d]pyrimidin-4-yl)hydrazono]methyl}-N-(2-pyrrolidin-1-ylethyl)benzamide

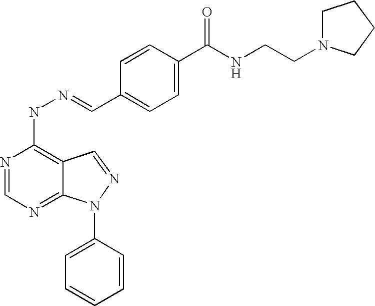

Prepared from 4-{(E)-[(1-phenyl-1H-pyrazolo[3,4-d]pyrimidin-4-yl)hydrazono]methyl}benzoic acid and 2-pyrrolidin-1-ylethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.35 (s, 1H), 8.66 (s, 1H), 8.52 (t, 1H), 8.49 (s, 1H), 8.32 (s, 1H), 8.20 (d, 2H), 7.90 (m, 4H), 7.55 (t, 2H), 7.35 (t, 1H), 3.30 (m, 6H), 2.55 (t, 2H), 1.65 (m, 4H). ES-MS m/z 455 (MH⁺).

### Example 35

### 4-({4-[2-(Dimethylamino)ethyl]piperazin-1-yl}carbonyl)benzaldehyde (1-phenyl-1H-pyrazolo[3,4-d]pyrimidin-4-yl)hydrazone

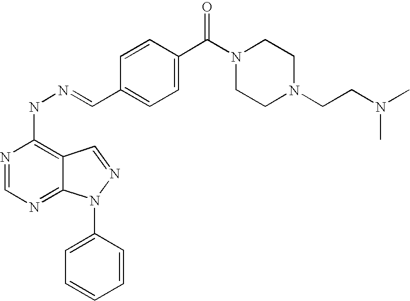

Prepared from 4-{(E)-[(1-phenyl-1H-pyrazolo[3,4-d]pyrimidin-4-yl)hydrazono]methyl}benzoic acid and N,N-dimethyl-2-piperazin-1-ylethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.31 (s, 1H), 8.65 (s, 1H), 8.48 (s, 1H), 8.31 (s, 1H), 8.20 (d, 2H), 7.87 (d, 2H), 7.55 (t, 2H), 7.47 (d, 2H), 7.35 (t, 1H), 3.58 (m, 2H), 2.47 (m, 4H), 2.38 (m, 4H), 2.31 (m, 2H), 2.09 (s, 6H). ES-MS m/z 498 (MH⁺).

### Example 36

### 4-{[4-(2-methoxyethyl)piperazin-1-yl]carbonyl}benzaldehyde (1-phenyl-1H-pyrazolo[3,4-d]pyrimidin-4-yl)hydrazone

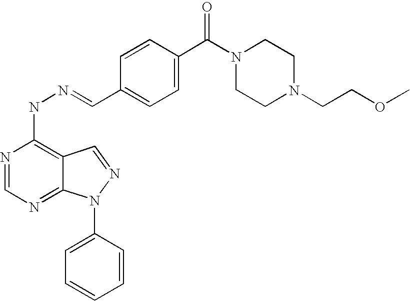

Prepared from 4-{(E)-[(1-phenyl-1H-pyrazolo[3,4-d]pyrimidin-4-yl)hydrazono]methyl}benzoic acid and 1-(2-methoxyethyl)piperazine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.34 (s, 1H), 8.65 (s, 1H), 8.49 (s, 1H), 8.31 (s, 1H), 8.20 (d, 2H), 7.87 (d, 2H), 7.55 (t, 2H), 7.47 (d, 2H), 7.35 (t, 1H), 3.59 (m, 2H), 3.41 (m, 2H), 3.20 (s, 3H), 2.47 (m, 8H). ES-MS m/z 485 (MH⁺).

### Example 37

### N[3-(4-Methylpiperazin-1-yl)propyl]-4-{(E)-[(1-phenyl-1H-pyrazolo[3,4-d]pyrimidin-4-yl)hydrazono]methyl}benzamide

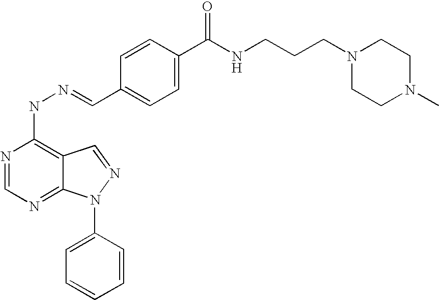

Prepared from 4-{(E)-[(1-phenyl-1H-pyrazolo[3,4-d]pyrimidin-4-yl)hydrazono]methyl}benzoic acid and 3-(4-methylpiperazin-1-yl)propan-1-amine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.34 (s, 1H), 8.66 (s, 1H), 8.58 (m, 1H), 8.49 (s, 1H), 8.33 (s, 1H), 8.20 (d, 2H), 7.90 (m, 4H), 7.56 (t, 2H), 7.35 (t, 1H), 3.29 (m, 6H), 2.31 (m, 6H), 2.12 (s, 3H), 1.66 (m, 2H). ES-MS m/z 498 (MH⁺).

### Example 38

### N-[2-(Methylsulfonyl)ethyl]-4-{(E)-[(1-phenyl-1H-pyrazolo[3,4-d]pyrimidin-4-yl)hydrazono]methyl}benzamide

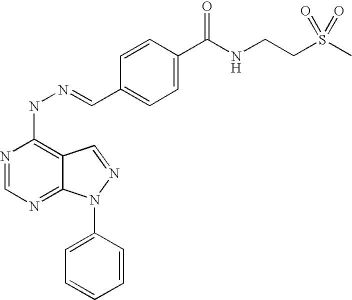

Prepared from 4-{(E)-[(1-phenyl-1H-pyrazolo[3,4-d]pyrimidin-4-yl)hydrazono]methyl}benzoic acid and 2-(methylsulfonyl)ethanamine using the method described by 4-{(E)-[(1-phenyl-1H-pyrazolo[3,4-d]pyrimidin-4-yl)hydrazono]methyl}-N-(3-pyrrolidin-1-ylpropyl)benzamide.

¹H NMR (400 MHz, DMSO) δ 12.37 (s, 1H), 8.81 (m, 1H), 8.67 (s, 1H), 8.50 (s, 1H), 8.33 (s, 1H), 8.20 (d, 2H), 7.92 (m, 4H), 7.56 (t, 2H), 7.35 (t, 1H), 3.66 (m, 2H), 3.38 (m, 2H), 3.02 (s, 3H). ES-MS m/z 464 (MH⁺).

### Example 39

### 3-{(E)-[(1-phenyl-1H-pyrazolo[3,4-d]pyrimidin-4-yl)hydrazono]methyl}benzoic Acid

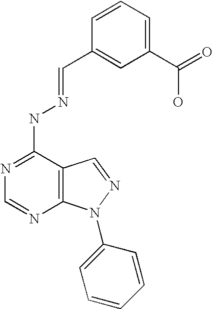

Prepared from 4-hydrazino-1-phenyl-1H-pyrazolo[3,4-d]pyrimidine and 3-formylbenzoic acid using the general procedure for isonicotinaldehyde (1-phenyl-1H-pyrazolo[3,4-d]pyrimidin-4-yl)hydrazone.

¹H NMR (300 MHz, DMSO) δ 12.76-12.05 (s br, 1H), 8.64 (s, 1H), 8.52 (t, 1H), 8.39 (s, 1H), 8.32 (s, 1H), 8.29-8.18 (m, 2H), 8.17-8.07 (m, 1H), 8.01 (d, 1H), 7.69-7.53 (m, 3H), 7.42-7.34 (m, 1H); ES-MS m/z 358 (MH⁺).

### Example 40

### N-[2-(dimethylamino)ethyl]-3-{(E)-[(1-phenyl-1H-pyrazolo[3,4-d]pyrimidin-4-yl)hydrazono]methyl}benzamide

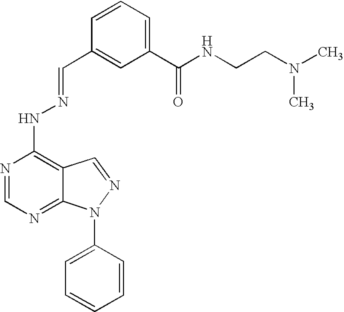

To a solution of 4-{(E)-[(1-phenyl-1H-pyrazolo[3,4-d]pyrimidin-4-yl)hydrazono]methyl}benzoic acid (43 mg, 0.12 mmol) in DMF (4 ml), was added N,N-dimethylethane-1,2-diamine (29 mg, 0.180 mmol), diethylcyanophosphonate (0.036 ml, 0.240 mmol), and triethylamine (0.05 ml, 0.360 mmol). The solution was stirred at rt for 3 h, then water and diethyl ether were added. The resulting percipitate was collected by filtration to give pure product (14 mg, yield 27%).

¹H NMR (300 MHz, DMSO) δ 12.34 (s, 1H), 8.69 (s, 1H), 8.59 (t, 1H), 8.51 (s, 1H), 8.36 (s, 1H), 8.22 (t, 3H), 8.00 (d, 1H), 7.95-7.86 (m, 1H), 7.63-7.53 (m, 3H), 7.41-7.34 (m, 1H), 3.45-3.35 (m, 2H), 2.53-2.43 (m, 2H), 2.22 (s, 6H); ES-MS m/z 429 (MH⁺).

### Example 41

### N-[2-(methylsulfonyl)ethyl]-3-{(1-[(1-phenyl-1H-pyrazolo[3,4-d]pyrimidin-4-yl)hydrazonolmethyl}benzamide

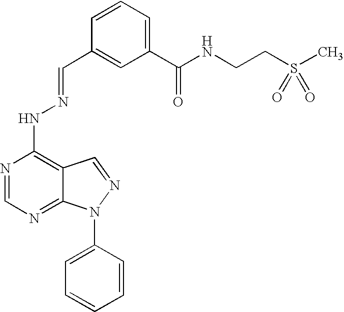

To a solution of 4-{(E)-[(1-phenyl-1H-pyrazolo[3,4-d]pyrimidin-4-yl)hydrazono]methyl}benzoic acid (43 mg, 0.12 mmol) in DMF (4 ml), was added 2-(methylsulfonyl)ethanamine (29 mg, 0.180 mmol), diethylcyanophosphonate (0.036 ml, 0.240 mmol), and triethylamine (0.05 ml, 0.360 mmol). The solution was stirred at rt for 3 h, then water and diethyl ether were added. The resulting percipitate was collected by filtration to give pure product (45 mg, yield 81%).

¹H NMR (300 MHz, DMSO) δ 12.37 (s, 1H), 8.92 (t, 1H), 8.70 (s, 1H), 8.52 (s, 1H), 8.37 (s, 1H), 8.22 (d, 3H), 8.05 (d, 1H), 7.90 (d, 1H), 7.68-7.53 (m, 3H), 7.41-7.34 (m, 1H), 3.73 (q, 2H), 3.42 (t, 2H), 3.07 (s, 3H); ES-MS m/z 464 (MH⁺).

### Example 42

### N-(2-morpholin-4-ylethyl)-3-{(E)-[(1-phenyl-1H-pyrazolo[3,4-d]pyrimidin-4-yl)hydrazono]methyl}benzamide

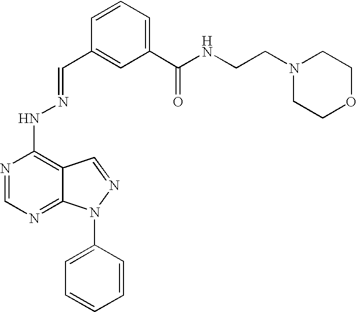

To a solution of 4-{(E)-[(1-phenyl-1H-pyrazolo[3,4-d]pyrimidin-4-yl)hydrazono]methyl}benzoic acid (43 mg, 0.12 mmol) in DMF (4 ml), was added 2-piperazin-1-ylethanamine (29 mg, 0.180 mmol), diethylcyanophosphonate (0.036 ml, 0.240 mmol), and triethylamine (0.05 ml, 0.360 mmol). The solution was stirred at rt for 3 h, then water and diethyl ether were added. The resulting percipitate was collected by filtration to give pure product (37 mg, yield 66%).

¹H NMR (300 MHz, DMSO) δ 12.36 (s, 1H), 8.69 (s, 1H), 8.61 (t, 1H), 8.52 (s, 1H), 8.37 (s, 1H), 8.25-8.20 (m, 3H), 8.01 (d, 1H), 7.88 (d, 1H), 7.62-7.53 (m, 3H), 7.38 (t, 1H), 3.63-3.55 (m, 4H), 3.44-3.40 (m, 2H), 2.50-2.47 (m, 2H), 2.46-2.39 (m, 4H); ES-MS m/z 471 (MH⁺).

### Example 43

### 4-Hydroxy-3-methoxybenzaldehyde (1-phenyl-1H-pyrazolo[3,4-d]pyrimidin-4-yl)hydrazone

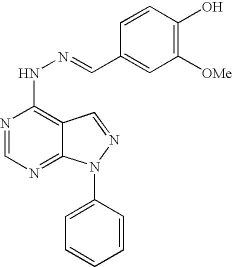

Prepared from 4-hydrazino-1-phenyl-1H-pyrazolo[3,4-d]pyrimidine (100 mg) and vanillan (67 mg) using the general procedure for isonicotinaldehyde (1-phenyl-1H-pyrazolo[3,4-d]pyrimidin-4-yl)hydrazone.

¹H NMR (300 MHz, DMSO) δ 8.64 (s, 1H), 8.50 (s, 1H), 8.33 (m, 3H), 7.60 (m, 2H), 7.39 (m, 2H), 7.26 (d, 1H), 6.94 (d, 1H) 3.94 (s, 3H). ES-MS m/z 361 (MH⁺).

### Example 44

### 4-Hydroxy-3-hydroxymethylbenzaldehyde (1-phenyl-1H-pyrazolo[3,4-d]pyrimidin-4-yl)hydrazone

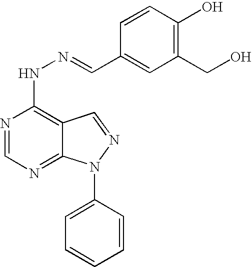

Prepared from 4-hydrazino-1-phenyl-1H-pyrazolo[3,4-d]pyrimidine (90 mg) and 4-Hydroxy-3-hydroxymethylbenzaldehyde (54 mg) using the general procedure for isonicotinaldehyde (1-phenyl-1H-pyrazolo[3,4-d]pyrimidin-4-yl)hydrazone.

¹H NMR (300 MHz, DMSO) δ 8.67 (s, 1H), 8.47 (s, 1H), 8.28 (s, 2H), 8.25 (s, 1H), 7.84 (s, 1H), 7.56 (m, 4H), 7.38 (m, 1H), 6.96 (d, 1H) 4.56 (s, 2H). ES-MS m/z 361 (MH⁺).

### Example 45

### 3-Hydroxy-4-methoxybenzaldehyde (1-phenyl-1H-pyrazolo[3,4-d]pyrimidin-4-yl)hydrazone

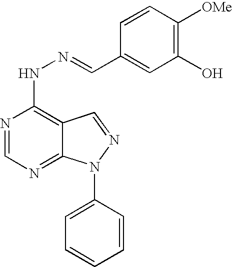

Prepared from 4-hydrazino-1-phenyl-1H-pyrazolo[3,4-d]pyrimidine (50 mg) and 3-hydroxy-4-methoxybenzaldehyde (34 mg) using the general procedure for isonicotinaldehyde (1-phenyl-1H-pyrazolo[3,4-d]pyrimidin-4-yl)hydrazone.

¹H NMR (300 MHz, DMSO) δ 9.52 (bs, 1H), 8.69 (s, 1H), 8.49 (s, 1H), 8.28 (s, 1H), 8.25 (s, 1H), 8.19 (s, 1H), 7.61 (m, 2H), 7.50 (s, 1H), 7.40 (m, 1H), 7.09 (d, 1H) 7.02 (m, 1H), 3.84 (s, #H). ES-MS m/z 361 (MH⁺).

### Example 46

### 3-Bromo-4-hydroxy-3-ethoxybenzaldehyde (1-phenyl-1H-pyrazolo[3,4-d]pyrimidin-4-yl)hydrazone

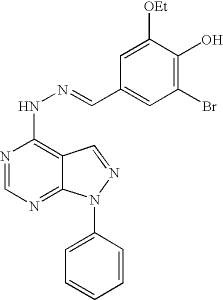

Prepared from 4-hydrazino-1-phenyl-1H-pyrazolo[3,4-d]pyrimidine (50 mg) and 3-bromo-4-hydroxy-5-ethoxybenzaldehyde (54 mg) using the general procedure for isonicotinaldehyde (1-phenyl-1H-pyrazolo[3,4-d]pyrimidin-4-yl)hydrazone.

¹H NMR (300 MHz, DMSO) δ 8.60 (s, 1H), 8.51 (s, 1H), 8.28 (d, 2H), 8.20 (s, 1H), 7.60 (m, 2H), 7.53 (s, 1H), 7.40 (m, 2H), 4.45 (q, 2H) 1.48 (t, 3H). ES-MS m/z 453, 454 (MH⁺).

### Example 47

### 3-Carboxy-4-Hydroxy-5-methoxybenzaldehyde (1-phenyl-1H-pyrazolo[3,4-d]pyrimidin-4-yl)hydrazone

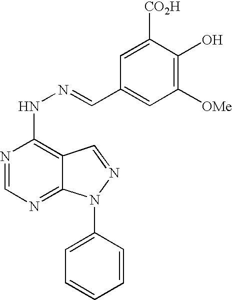

Prepared from 4-hydrazino-1-phenyl-1H-pyrazolo[3,4-d]pyrimidine (50 mg) and 5-carboxyvanillin (43 mg) using the general procedure for isonicotinaldehyde (1-phenyl-1H-pyrazolo[3,4-d]pyrimidin-4-yl)hydrazone.

¹H NMR (300 MHz, DMSO) δ 12.15 (bs, 1H), 8.64 (s, 1H), 8.48 (s, 1H), 8.28 (m, 3H), 7.72 (s, 1H), 7.59 (m, 2H), 7.50 (s, 1H), 7.38 (m, 1H), 3.87 (s, 3H). ES-MS m/z 405 (MH⁺).

### Example 48

### 3-Bromo-4-hydroxy-3-methoxybenzaldehyde (1-phenyl-1H-pyrazolo[3,4-d]pyrimidin-4-yl)hydrazone

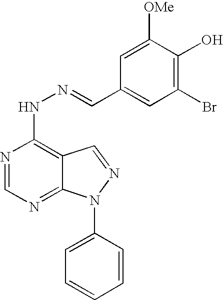

Prepared from 4-hydrazino-1-phenyl-1H-pyrazolo[3,4-d]pyrimidine (50 mg) and 3-bromo-4-hydroxy-5-methoxybenzaldehyde (51 mg) using the general procedure for isonicotinaldehyde (1-phenyl-1H-pyrazolo[3,4-d]pyrimidin-4-yl)hydrazone.

¹H NMR (300 MHz, DMSO) δ 8.60 (s, 1H), 8.48 (s, 1H); 8.25 (d, 2H), 8.18 (s, 1H), 7.60 (m, 2H), 7.46 (s, 1H), 7.39 (m, 2H), 3.98 (s, 3H). ES-MS m/z 440, 441 (MH⁺).

### Example 49

### 3-Ethoxy-4-hydroxy-benzaldehyde (1-phenyl-1H-pyrazolo[3,4-d]pyrimidin-4-yl)hydrazone

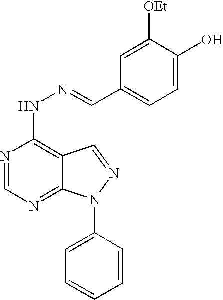

Prepared from 4-hydrazino-1-phenyl-1H-pyrazolo[3,4-d]pyrimidine (30 mg) and 4-hydroxy-3-ethoxybenzaldehyde (22 mg) using the general procedure for isonicotinaldehyde (1-phenyl-1H-pyrazolo[3,4-d]pyrimidin-4-yl)hydrazone.

¹H NMR (300 MHz, DMSO) δ 8.62 (s, 1H), 8.48 (s, 1H), 8.22 (m, 3H), 7.60 (m, 2H), 7.38 (m, 2H), 7.23 (m, 1H), 6.84 (d, 1H), 4.20 (q, 2H) 1.41 (t, 3H) ES-MS m/z 375 (MH⁺).

### Example 50

### 4-Fluorobenzaldehyde (1-phenyl-1H-pyrazolo[3,4-d]pyrimidin-4-yl)hydrazone

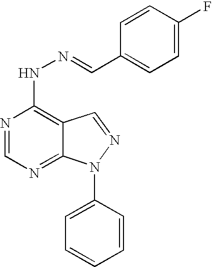

Prepared from 4-hydrazino-1-phenyl-1H-pyrazolo[3,4-d]pyrimidine (30 mg) and 4-fluorobenzaldehyde (22 mg) using the general procedure for isonicotinaldehyde (1-phenyl-1H-pyrazolo[3,4-d]pyrimidin-4-yl)hydrazone.

¹H NMR (300 MHz, DMSO) δ 8.69 (s, 1H), 8.52 (s, 1H), 8.34 (s, 1H), 8.25 (d, 2H), 7.96 (m, 2H), 7.61 (m, 2H), 7.37 (m, 3H). ES-MS m/z 333 (MH⁺).

### Example 51

### 5-Hydroxymethyl-2-furaldehyde [1-(phenyl)-1H-pyrazolo[3,4-d]pyrimidin-4-yl]hydrazone

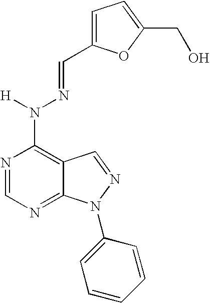

To a stirred solution of 4-hydrazino-1-(phenyl)-1H-pyrazolo[3,4-d]pyrimidine (50 mg, 0.22 mmol) in ethanol (5 ml) was added 5-hydroxymethyl-2-furaldehyde (28 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (13 mg, 18%).

¹H NMR (DMSO) δ 12.2 (br s, 1H), 8.62 (s, 1H), 8.43 (s, 1H), 8.21 (d, 2H), 8.11 (s, 1H), 7.55 (t, 2H), 7.34 (t, 1H), 6.93 (d, 1H), 6.46 (d, 1H), 5.42 (br s, 1H), 4.49 (s, 2H).

### Example 52

### 4-Acetamidobenzaldehyde [1-(phenyl)-1H-pyrazolo[3,4-d]pyrimidin-4-yl]hydrazone

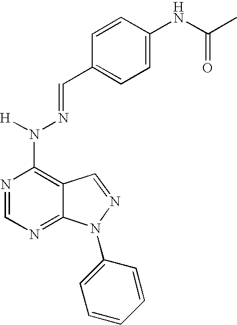

To a stirred solution of 4-hydrazino-1-(phenyl)-1H-pyrazolo[3,4-d]pyrimidine (50 mg, 0.22 mmol) in ethanol (5 ml) was added 4-acetamidobenzaldehyde (36 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (46 mg, 56%).

¹H NMR (DMSO) δ 12.20 (br s, 1H), 10.16 (s, 1H), 8.65 (s, 1H), 8.42 (s, 1H), 8.22 (m, 3H), 7.72 (m, 4H), 7.55 (t, 2H), 7.34 (t, 1H), 2.06 (s, 3H).

### Example 53

### 4-(2-Hydroxyethoxy)benzaldehyde [1-(phenyl)-1H-pyrazolo[3,4-d]pyrimidin-4-yl]hydrazone

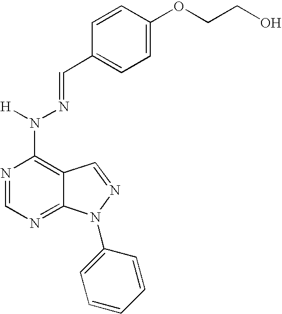

To a stirred solution of 4-hydrazino-1-(phenyl)-1H-pyrazolo[3,4-d]pyrimidine (50 mg, 0.22 mmol) in ethanol (5 ml) was added 4-(2-hydroxyethoxy)benzaldehyde (37 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (44 mg, 54%).

¹H NMR (DMSO) δ 12.10 (br s, 1H), 8.62 (s, 1H), 8.41 (s, 1H), 8.21 (s, 1H), 7.76 (d, 2H), 7.55 (t, 2H), 7.34 (t, 1H), 7.04 (d, 2H), 4.92 (br s, 1H), 4.04 (t, 2H), 3.73 (t, 2H).

### Example 54

### 3-Hydroxy-4-methoxybenzaldehyde [1-(phenyl)-1-pyrazolo[3,4-d]pyrimidin-4-yl]hydrazone

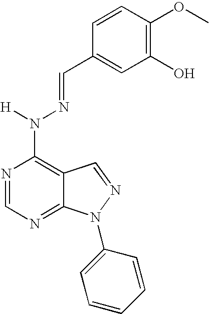

To a stirred solution of 4-hydrazino-1-(phenyl)-1H-pyrazolo[3,4-d]pyrimidine (50 mg, 0.22 mmol) in ethanol (5 ml) was added 3-hydroxy-4-methoxybenzaldehyde (34 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (34 mg, 43%).

¹H NMR (DMSO) δ 8.63 (s, 1H), 8.36 (s, 1H), 8.23 (d, 2H), 8.13 (s, 1H), 7.54 (t, 2H), 7.45 (s, 1H), 7.33 (t, 1H), 7.00 (m, 2H).

### Example 55

### 3-Methoxy-4-hydroxybenzaldehyde [1-(phenyl)-1H-pyrazolo[3,4-d]pyrimidin-4-yl]hydrazone

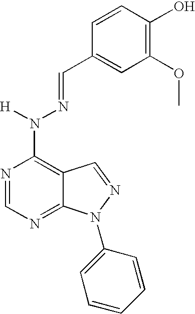

To a stirred solution of 4-hydrazino-1-(phenyl)-1H-pyrazolo[3,4-d]pyrimidine (50 mg, 0.22 mmol) in ethanol (5 ml) was added 3-methoxy-4-hydroxybenzaldehyde (34 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (41 mg, 52%).

¹H NMR (DMSO) δ 8.59 (s, 1H), 8.42 (s, 1H), 8.20 (m, 3H), 7.55 (t, 2H), 7.34 (m, 2H), 7.19 (d, 1H), 6.85 (d, 1H).

### Example 56

### 3,4-Dimethoxybenzaldehyde [1-(phenyl)-1H-pyrazolo[3,4-d]pyrimidin-4-yl]hydrazone

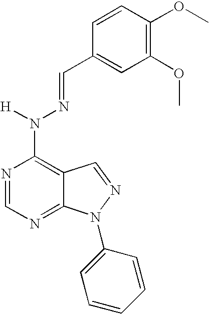

To a stirred solution of 4-hydrazino-1-(phenyl)-1H-pyrazolo[3,4-d]pyrimidine (50 mg, 0.22 mmol) in ethanol (5 ml) was added 3,4-dimethoxy benzaldehyde (37 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (34 mg, 41%).

¹H NMR (DMSO) δ 12.21 (br s, 1H), 8.58 (s, 1H), 8.42 (s, 1H), 8.22 (m, 3H), 7.55 (t, 2H), 7.32 (m, 3H), 7.04 (d, 1H), 3.87 (s, 3H), 3.80 (s, 3H).

### Example 57

### 5-Ethyl-2-furaldehyde [1-(phenyl)-1H-pyrazolo[3,4-d]pyrimidin-4-yl]hydrazone

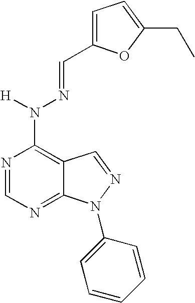

To a stirred solution of 4-hydrazino-1-(phenyl)-1H-pyrazolo[3,4-d]pyrimidine (50 mg, 0.22 mmol) in ethanol (5 ml) was added 5-ethyl-2-furaldehyde (27 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (27 mg, 37%).

¹H NMR (DMSO) δ 12.20 (br s, 1H), 8.59 (s, 1H), 8.42 (s, 1H), 8.21 (d, 2H, J=7.9 Hz), 8.07 (s, 1H), 7.55 (t, 2H), 7.34 (t, 1H), 6.89 (d, 1H), 6.29 (d, 1H), 2.73 (q, 2H), 1.24 (t, 3H).

### Example 58

### Pyridine-N-oxide-4-carboxaldehyde [1-(phenyl)-1H-pyrazolo[3,4-d]pyrimidin-4-yl]hydrazone

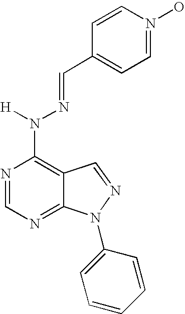

To a stirred solution of 4-hydrazino-1-(phenyl)-1H-pyrazolo[3,4-d]pyrimidine (50 mg, 0.22 mmol) in ethanol (5 ml) was added pyridine-N-oxide-4-carboxaldehyde (27 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (47 mg, 64%).

¹H NMR (DMSO) δ 12.45 (br s, 1H), 8.66 (s, 1H), 8.47 (s, 1H), 8.22 (m, 5H), 7.83 (d, 2H), 7.56 (t, 2H), 7.35 (t, 1H).

### Example 59

### 5-Methyl-2-furaldehyde [1-(phenyl)-1H-pyrazolo[3,4-d]pyrimidin-4-yl]hydrazone

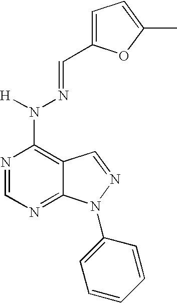

To a stirred solution of 4-hydrazino-1-(phenyl)-1H-pyrazolo[3,4-d]pyrimidine (50 mg, 0.22 mmol) in ethanol (5 ml) was added 5-methyl-2-furaldehyde (24 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (27 mg, 39%).

¹H NMR (DMSO) δ 12.16 (br s, 1H), 8.58 (s, 1H), 8.39 (s, 1H), 8.22 (d, 2H), 8.06 (s, 1H), 7.54 (t, 2H), 7.33 (t, 1H), 6.86 (d, 1H), 6.27 (d, 1H), 2.39 (s, 3H).

### Example 60

### 4-(2-(Diethylamino)-ethoxy)-benzaldehyde [1-(phenyl)-1H-pyrazolo[3,4-d]pyrimidin-4-yl]hydrazone

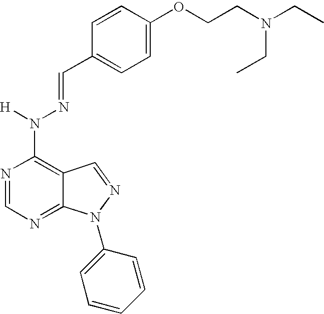

To a stirred solution of 4-hydrazino-1-(phenyl)-1H-pyrazolo[3,4-d]pyrimidine (50 mg, 0.22 mmol) in ethanol (5 ml) was added 4-(2-(diethylamino)-ethoxy)-benzaldehyde (49 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (28 mg, 30%).

¹H NMR (DMSO) δ 12.10 (br s, 1H), 8.61 (s, 1H), 8.45 (s, 1H), 8.23 (m, 3H), 7.74 (d, 2H), 7.55 (t, 2H), 7.35 (t, 1H), 7.03 (d, 2H), 4.05 (t, 2H), 2.77 (t, 2H), 2.53 (q, 4H), 0.96 (t, 6H).

### Example 61

### 4-Formylcin namic acid [1-(phenyl)-1H-pyrazolo[3,4-d]pyrimidin-4-yl]hydrazone

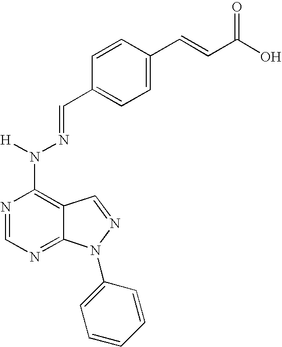

To a stirred solution of 4-hydrazino-1-(phenyl)-1H-pyrazolo[3,4-d]pyrimidine (50 mg, 0.22 mmol) in ethanol (5 ml) was added 4-formylcinnamic acid (39 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (30 mg, 35%).

¹H NMR (DMSO) δ 8.66 (s, 1H), 8.49 (s, 1H), 8.31 (s, 1H), 8.21 (d, 2H), 7.82 (d, 2H), 7.70 (d, 2H), 7.57 (t, 2H), 7.36 (m, 2H), 6.54 (d, 1H), 3.38 (br s, 1H).

### Example 62

### 4-Quinolinecarboxaldehyde [1-(phenyl)-1H-pyrazolo[3,4-d]pyrimidin-4-yl]hydrazone

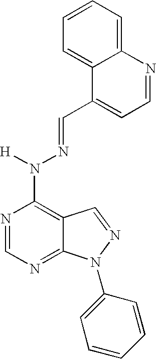

To a stirred solution of 4-hydrazino-1-(phenyl)-1H-pyrazolo[3,4-d]pyrimidine (50 mg, 0.22 mmol) in ethanol (5 ml) was added 4-quinolinecarboxaldehyde (35 mg, 0.22 mmol) and pyrrolidine (2 drops). The resulting mixture was heated at 78° C. for 13 h and cooled to room temperature. The resulting solids were filtered and washed with cold ethanol to yield the product as an off-white solid (22 mg, 28%).

¹H NMR (DMSO) δ 12.54 (s, 1H), 9.03 (m, 2H), 8.66 (s, 1H), 8.54 (s, 1H), 8.38 (m, 1H), 8.20 (m, 2H), 8.09 (m, 2H), 7.79 (m, 2H), 7.54 (m, 2H), 7.37 (m, 1H).

Biologigal Data

GSK3

The compounds of the present invention elicit important and measurable pharmacological responses. In evaluating those responses, the present invention also demonstrated unexpected advantageous biological and pharmacological properties. In short, the present invention provides unexpected superior performance characteristics not heretofore appreciated.

The protocol used to demonstrate the pharmacological response of the present invention is based on the ability of the kinase to phosphorylate a biotinylated peptide, the sequence of which is derived from the phosphorylation site of glycogen synthase and its sequence is: Biotin-Ahx-AAAKRREILSRRPS(PO₃)YR-amide. The phosphorylated biotinylated peptide is then captured onto streptavidin coated scintillation proximity assay (SPA) beads from Amersham Technology, where the signal from the ³³P is amplified via the scintillant contained in the beads.

GSK-3β is commercially available or may be cloned and expressed in E. coli using standard techniques to produce soluble, active protein. The production of active protein involves purification in two steps using Metal Chelate and Ion Exchange Chromatography. Protein eluting from Ion Exchange provides >90% pure product that may then be concentrated for use in high throughput screening.

The kinase was assayed at a concentration of 20 nM final in 100 mM HEPES, pH 7.2 containing 10 mM magnesium chloride, 0.1 mg/mL bovine serum albumin, 1 mM dithiothreitol, 0.3 mg/mL heparin, 2.8 uM peptide substrate, 2.5 uM ATP, and 0.2 uCi/well [□-³³P]-ATP. After 40 minutes incubation at room temperature, the reaction was stopped by addition of 100 mM EDTA and 1 mM solution in 100 mM HEPES, pH7.2 followed by an additional solution of diluted Streptavidin coated SPA beads in PBS, pH 7.2 to give a final concentration of 0.25 mg of beads per assay well in a 96-well microtiter plate.

10 mM stock solutions of the compounds of the invention in 100% DMSO are generated as a first step in the screening process. The second step involves the creation of dose response plates where these compounds are diluted 10-fold in 100% DMSO to 1 mM concentrations and subsequently serially diluted 3-fold in 100% DMSO across the plate by automated liquid handling such that the final top concentration of inhibitor is 0.033 mM in the 30 uL kinase assay. The third step involves the creation of the assay plates. This is achieved by transferring 1 uL of the compounds to assay plates by automated liquid handling. The fourth step is to perform the assay as described and count the resulting plates in the Packard TopCount NXT microplate scintillation and luminescence counter.

The final step is data acquisition and analysis where IC₅₀ values are generated for each compound by normalizing curve data to the equation 100*(U1−C2)/(C1−C2) (where U1 is the cpm value, C2 is the background, and C1 is the maximum number of counts), then fitting the normalized data to the equation y=Vmax*(1−(x/(K+x))). The IC₅₀ values were converted to pIC₅₀ values, i.e., −log IC₅₀ in Molar concentration. The data is expressed below in Table 1.

TABLE 1

| Example # | GSK-3 pIC₅₀ |
|---|---|
| 1 | ++ |
| 2 | +++ |
| 3 | ++ |
| 4 | ++ |
| 5 | +++ |
| 6 | ++ |
| 7 | +++ |
| 8 | ++ |
| 9 | ++ |
| 10 | +++ |
| 12 | ++ |
| 13 | + |
| 14 | ++ |
| 15 | ++ |
| 16 | +++ |
| 17 | ++ |
| 18 | ++ |
| 19 | +++ |
| 20 | +++ |
| 21 | +++ |
| 22 | +++ |
| 23 | +++ |
| 24 | ++ |
| 25 | +++ |
| 26 | +++ |
| 27 | +++ |
| 28 | ++ |
| 29 | ++ |
| 30 | ++ |
| 31 | ++ |
| 32 | ++ |
| 33 | +++ |
| 34 | ++ |
| 35 | ++ |
| 36 | ++ |
| 37 | ++ |
| 38 | +++ |
| 39 | + |
| 40 | +++ |
| 41 | ++ |
| 42 | ++ |
| 43 | +++ |
| 44 | ++ |
| 45 | ++ |
| 46 | + |
| 47 | ++ |
| 48 | ++ |
| 49 | + |
| 50 | ++ |
| 51 | + |
| 52 | ++ |
| 53 | ++ |
| 54 | ++ |
| 55 | +++ |
| 56 | ++ |
| 57 | + |
| 58 | +++ |
| 59 | ++ |
| 60 | ++ |
| 61 | ++ |
| 62 | + |

+ = pIC₅₀ of 5.0-6.0;
++ = pIC₅₀ of 6.0-7.0;
+++ = pIC₅₀ of >7.0.

TIE-2 Enzyme Assay (TIE2-E)

The TIE-2 enzyme assay used the LANCE method (Wallac) and GST-TIE2, baculovirus expressed recombinant constructs of the intracellular domains of human TIE2 (amino acids 762-1104, GenBank Accession # L06139) tagged by GST). The method measured the ability of the purified enzymes to catalyse the transfer of the γ-phosphate from ATP onto tyrosine residues in a biotinylated synthetic peptide, D1-15 (biotin-C6-LEARLVAYEGWVAGKKKamide). This peptide phosphorylation was detected using the following procedure: for enzyme preactivation, GST-TIE2 was incubated for 30 mins at room temperature with 2 mM ATP, 5 mM MgCl2 and 12.5 mM DTT in 22.5 mM HEPES buffer (pH7.4). Preactivated GST-TIE2 was incubated for 30 mins at room temperature in 96 well plates with 1 μM D1-15 peptide, 80 uM ATP, 10 mM MgCl₂, 0.1 mg/ml BSA and the test compound (diluted from a 10 mM stock in DMSO, final DMSO concentration was 2.4%) in 1 mM HEPES (pH7.4). The reaction was stopped by the addition of EDTA (final concentration 45 mM). Streptavidin linked-APC (allophycocyanin, Molecular Probe) and Europium-labeled anti-phosphorylated tyrosine antibody (Wallac) were then added at the final concentration of 17 μg/well and 2.1 μg/well, respectively. The APC signal was measured using an ARVO multilabel counter. (Wallac Berthold Japan). The percent inhibition of activity was calculated relative to blank control wells.

The concentration of test compound that inhibits 50% of activity (IC₅₀) was interpolated using nonlinear regression (Levernberg-Marquardt) and the equation, y=Vmax (1−x/(K+x))+Y2, where “K” was equal to the IC₅₀. The IC₅₀ values were converted to pIC₅₀ values, i.e., −log IC₅₀ in Molar concentration. The results are represented in Table 2 below.

Test compounds are employed in free or salt form.

TABLE 2

| Example # | TIE2-E |
|---|---|
| 1 | ++ |
| 2 | + |
| 5 | ++ |
| 6 | + |
| 7 | ++ |
| 20 | ++ |
| 24 | ++ |
| 25 | ++ |
| 26 | ++ |
| 27 | ++ |
| 29 | ++ |
| 30 | ++ |
| 31 | + |
| 32 | ++ |
| 33 | ++ |
| 34 | ++ |
| 35 | ++ |
| 36 | ++ |
| 37 | ++ |
| 38 | ++ |
| 39 | + |
| 43 | ++ |
| 44 | ++ |
| 45 | ++ |
| 46 | ++ |
| 47 | + |
| 48 | ++ |
| 49 | + |
| 50 | + |
| 51 | + |
| 52 | ++ |
| 53 | + |
| 54 | ++ |
| 55 | ++ |
| 56 | ++ |
| 58 | + |
| 60 | ++ |
| 61 | ++ |

+ = pIC₅₀ of 5.0-6.0;
++ = pIC₅₀ of 6.0-7.0;
+++ = pIC₅₀ of >7.0.

All research complied with the principles of laboratory animal care (NIH publication No. 85-23, revised 1985) and GlaxoSmithKline policy on animal use.

Although specific embodiments of the present invention have been illustrated and described in detail, the invention is not limited thereto. The above detailed description of preferred embodiments is provided for example only and should not be construed as constituting any limitation of the invention. Modifications will be obvious to those skilled in the art, and all modifications that do not depart from the spirit of the invention are intended to be included within the scope of the appended claims.

1. A method for the treatment or prophylaxis of a disease or condition, said disease or condition characterized by misregulation of a protein kinase, comprising administering of a compound of Formula (I): 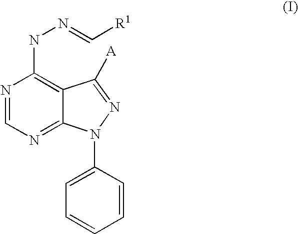
including salts, solvates, and pharmaceutically acceptable derivatives thereof,
wherein A is H, alkyl, or aryl;
R¹ is D¹, D², D³, D⁴, or D⁵,
wherein D¹ is 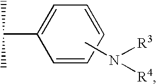
and R³ and R⁴ are each independently H, alkyl, alkylsulfonyl, or —C(O)—(CH₂)ₓ—R⁵,
where R⁵ is alkyl, acyl, alkoxy, —(O)—(CH₂)ₓ—(O)-alkyl, or —NR⁶R⁷,
where R⁶ and R⁷ are each independently H or alkyl, or
R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen,
or R³ and R⁴ combine to form a 5- or 6-membered ring, optionally containing one or more additional heteroatoms, optionally containing one or more degrees of unsaturation, and optionally substituted one or more times with alkyl, hydroxy, carboxy, alkoxy, acyl, or halogen;
wherein D² is 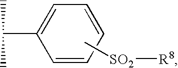
and R⁸ is alkyl, or —NR⁹R¹⁰,
where R⁹ and R¹⁰ are each independently selected from H, alkyl, or —(CH₂)ₓ—NR⁶R⁷,
where R⁶ and R⁷ are each independently H or alkyl,
or R⁶ and R⁷ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen;
wherein D³ is 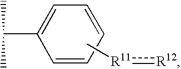
and
the dashed line represents an optional double bond;
when R¹¹ is —(CH₂)], the optional dashed double bond does not exist, and R¹² is alkylsulfonyl or —NR¹³R¹⁴
where R¹³ and R¹⁴ are each independently selected from H, alkyl, —(CH₂)ₓ—R¹⁷, where R¹⁷ is alkoxy or —NR¹⁵R¹⁶,
where R¹⁵ and R¹⁶ are each independently H or alkyl,
or R¹³ and R¹⁴ combine to form a 5- or 6-membered ring, optionally containing one or more additional heteroatoms, optionally containing one or more degrees of unsaturation, and optionally substituted one or more times with alkyl or —(CH₂)ₓ—OH;
when R¹¹ is —(CH)—, the optional dashed double bond exists, and R¹² is —(CH)—C(O)—OH;
wherein D⁴ is 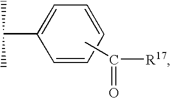
and R¹⁷ is hydroxy, alkoxy, or —NR¹⁸R¹⁹,
where R¹⁸ and R¹⁹ are each independently selected from H, alkyl, —(CH₂)ₙ—R²⁰,
where R²⁰ is alkylsulfonyl, hydroxy, aryl said aryl optionally substituted with hydroxy or alkoxy, heteroaryl, or —NR²¹R²²,
where R²¹ and R²² are each independently selected from H, acyl, alkyl,
or R²¹ and R²² combine to form a 5- or 6-membered ring, optionally containing one or more additional heteroatoms, optionally containing one or more degrees of unsaturation, and optionally substituted with alkyl or —(CH₂)ₙ—OH;
or R¹⁸ and R¹⁹ combine to form a 5- or 6-membered ring, optionally containing one or more additional heteroatoms, optionally containing one or more degrees of unsaturation, and optionally substituted with —(CH₂)ₓ—R²³,
where R²³ is alkoxy, hydroxy, —C(O)—R²⁴, where R²⁴ is a 5- or 6-membered ring optionally containing one or more heteroatoms and optionally containing one or more degrees of unsaturation, or —NR²⁵R²⁶, where R²⁵ and R²⁶ are each independently H or alkyl;
wherein D⁵ is
a 5- or 6-membered ring, optionally containing one or more heteroatoms, optionally containing one or more degrees of unsaturation, optionally fused with an additional 5- or 6-membered ring that optionally contains one or more heteroatoms and optionally contains one or more degrees of unsaturation,
wherein the ring or fused ring system may be optionally substituted one or more times with halogen, alkyl, haloalkyl, alkylsulfonyl, alkylthio, hydroxy, alkoxy, oxo, sulfonyl, sulfate ion, nitro, cyano, carboxy, alkoxycarbonyl, aryl where said aryl may be optionally substituted with sulfamoyl, heteroaryl where said heteroaryl may be optionally substituted with alkyl, or —NR²⁷R²⁸,
where R²⁷ and R²⁸ are each independently H, alkyl, acyl, alkoxy, alkoxycarbonyl, carboxy, or —(CH₂)ₓ—NR²⁹R³⁰ where R²⁹ and R³⁰ are each independently selected from H and alkyl,
or R²⁷ and R²⁸ combine to form a 5- or 6-membered ring, optionally containing one or more additional heteroatoms, optionally containing one or more degrees of unsaturation, and optionally substituted one or more times with alkyl, hydroxy, carboxy, acyl, alkoxy, or halogen,
or —(O)_{y}—(CH₂)ₓ—R³¹ where R³¹ is hydroxy, alkoxy, haloalkyl, aryl optionally substituted with halogen, or —NR²⁷R²⁸, where R²⁷ and R²⁸ are as defined above;
provided that if D⁵ is phenyl, said phenyl must be substituted
wherein for each occurrence, x independently is 0, 1, 2, or 3; and
wherein for each occurrence, y independently is 0 or 1.

2. The method of claim 1 wherein R¹ is D⁵; and
D⁵ is pyridyl substituted one or more times with alkoxy, halogen, —NR²⁷R²⁸,
where R²⁷ is H or alkyl, and
R²⁸ is H, alkyl, acyl, alkoxycarbonyl, or —(CH₂)ₓ—NR²⁹R³⁰,
where x is 2 and R²⁹ and R³⁰ are each alkyl, or —(O)_{y}—(CH)ₓ—R³¹,
where y is 1, x is 2, and R³¹ is —NR²⁷R²⁸, where R²⁷ and R²⁸ are each alkyl.

3. The method of claim 1 wherein R¹ is D⁵; and D⁵ is quinolinyl.

4. The method of claim 1 wherein R¹ is D⁵; and D⁵ is piperadinyl optionally substituted with alkoxycarbonyl.

5. The method of claim 1 wherein R¹ is D²; and
R⁸ is —NR⁹R¹⁰,
where R⁹ is H, and
R¹⁰ is H or —(CH₂)ₓ—NR⁶R⁷,
where x is 2 or 3, and
R⁶ and R⁷ are each alkyl or
R⁶ and R⁷ combine to form morpholinyl or pyrrolidinyl.

6. The method of claim 1 wherein R¹ is D⁴; and
R¹⁷ is hydroxy or —NR¹⁸R¹⁹
where R¹⁸ is H or alkyl, and
R¹⁹ is —(CH₂)ₓ—R²⁰,
where x is 2 or 3, and
R²⁰ is alkylsulfonyl, pyridyl, imidazolyl, or —NR²¹R²²,
where R²¹ and R²² are each H or alkyl, or
R²¹ and R²² combine to form piperidinyl, pyrrolidinyl, morpholinyl, or piperazinyl, each optionally substituted with alkyl, or
R¹⁸ and R¹⁹ combine to form piperizinyl optionally substituted with —(CH₂)ₓ—R²³,
where x is 2 and R²³ is alkoxy or —NR²⁵R²⁶,
where R²⁵ and R²⁶ are each alkyl.

7. The method of claim 1 wherein R¹ is D⁵; and
D⁵ is phenyl substituted one or more times with alkoxycarbonyl, hydroxy, halogen, alkoxy, carboxy, or —(O)_{y}—(CH₂)ₙ—R³¹,
where y is 0 or 1,
x is 1 or 2, and
R³¹ is hydroxy.

8. The method of claim 1 wherein the kinase is a serine/threosine kinase.

9. The method of claim 1 wherein the kinase is GSK3.

10. The method of claim 1 wherein the kinase is a tyrosine kinase.

11. The method of claim 1 wherein the kinase is TIE2.

12. The method of claim 1 wherein the disease or condition is type 2 diabetes, hyperlipidemia, obesity, CNS disorders, neurotraumatic injuries, immune potentiation, baldness or hair loss, atherosclerotic cardiovascular disease, hypertension, polycystic ovary syndrome, ischemia, immunodeficiency, or cancer.

13. The method of claim 1 wherein the disease or condition is type 2 diabetes and the method further comprises administering at least one additional anti-diabetic agent.

14-20. (canceled)
